(19) United States
(12) Patent Application Publication
(10) Pub. No.: US 2022/0125906 A1
(43) Pub. Date: Apr. 28, 2022

(54) TREATMENT OF BENIGN NERVOUS SYSTEM TUMORS USING ATTENUATED SALMONELLA TYPHIMURIUM

(71) Applicant: The General Hospital Corporation, Boston, MA (US)

(72) Inventors: Gary J. Brenner, Winchester, MA (US); Sherif Ahmed, Somerville, MA (US)

(21) Appl. No.: 17/434,349

(22) PCT Filed: Feb. 27, 2020
(86) PCT No.: PCT/US2020/020160

§ 371 Date: Aug. 26, 2021

Related U.S. Application Data
(60) Provisional application No. 62/811,066, filed on Feb. 27, 2019.

(51) Int. Cl. A61K 39/112; A61K 39/395; C07K 16/28; A61P 35/00

(52) U.S. Cl. CPC A61K 39/0275; A61K 39/39541; A61K 2039/522; A61P 35/00; C07K 16/2818

(57) ABSTRACT

Compositions and methods for the treatment of benign nervous system tumors including schwannomas using attenuated Salmonella typhimurium and optionally one or more checkpoint inhibitors.

### CLAIM OF PRIORITY

This application claims the benefit of U.S. Patent Application Ser. No. 62/811,066, filed on 27 Feb. 2019. The entire contents of the foregoing are hereby incorporated by reference.

### TECHNICAL FIELD

Provided herein are compositions and methods for the treatment of benign nervous system tumors including schwannomas using attenuated Salmonella typhimurium and optionally one or more checkpoint inhibitors.

### BACKGROUND

Schwannomas are slow-growing benign neoplasms derived from Schwann-lineage cells^{1,2}. Depending on location and size, these tumors can cause a variety of gain- and loss-of-function neurological deficits including hearing loss, imbalance, tinnitus, motor loss, and severe pain^{3,4}; in some cases they can lead to death due to brain stem compression⁵. Schwannomas may arise sporadically (thus, termed “sporadic schwannoma”) or as part of the debilitating genetic syndromes neurofibromatosis type 2 (NF2) and schwannomatosis⁶. Treatment of schwannoma is largely limited to operative resection and symptomatic management of pain. Resection, which for many patients is non-curative, is often associated with additional neurologic damage, and may be impractical due to location or large numbers of tumors⁷. Anti-cancer therapeutics have not demonstrated efficacy for schwannomas due to the slow replicating nature of these benign lesions^{8,9,10}. Bevacizumab is presently the only generally accepted pharmacotherapy for schwannoma; it temporally stabilizes tumor growth by targeting the highly vascularized nature of a subset of these neoplasms^{8,9,11}. Current strategies for pain control are, unfortunately, inadequate for many thus further increasing the burden of disease. Treatment is further complicated by the fact that schwannomas appear in multiple locations with new lesions developing throughout life. Thus, schwannomas- and their associated diseases cause lifelong suffering that cannot be stably controlled with current treatment options.

### SUMMARY

Schwannomas are slow-growing, benign neoplasms that develop throughout the body including along the spinal cord and within the cranium. Schwannomas frequently first appear in childhood or adolescence with new tumors developing throughout life. These tumors cause pain, sensory/motor dysfunction, and death through compression of peripheral nerves, the spinal cord, and/or the brain. The great suffering and debility associated with schwannomas, in conjunction with the paucity of therapeutic options makes their treatment a major unmet medical need. Described herein is a therapeutic approach for benign neoplasms including schwannoma that involves intratumoral (i.t.) injection of attenuated Salmonella typhimurium (S. typhimurium). The present results demonstrate the ability of this i.t. S. typhimurium to control tumor growth in both a xenograft human-NF2 schwannoma model in nude mice and in an allograft genetic mouse-schwannoma model in immune competent animals. Schwannoma growth control in the allograft model was associated with tumor cell apoptosis, decreased tumor angiogenesis, and induction of anti-tumor adaptive immune responses. I.t. S. typhimurium injection led to tumor control not only of bacterially-injected tumors but also of simultaneously developing distal schwannomas. Further, i.t. S. typhimurium controlled growth of re-challenge schwannomas implanted contralateral to the primary tumor 13-days following primary treatment. In an allograft schwannoma model, systemic application of a programmed death-1 receptor (PD-1) checkpoint inhibitor controlled tumor growth to the same degree as i.t. S. typhimurium, and combination of the two therapeutics had an additive effect on growth control in bacterially-injected and a synergistic effect on T-cell subset populations.

The present data support therapy via i.t. attenuated S. typhimurium, optionally in combination with PD-1 checkpoint inhibition, as an immunotherapy capable of controlling growth of bacterially-injected and non-injected benign nervous system tumors including schwannomas and schwannoma-related neoplasms including NF1-associated tumors and meningiomas. The presented data further suggest the potential of the therapeutic strategy to control growth of tumors that arise following initial treatment. Importantly, direct injection of attenuated Salmonella typhimurium into tumors had a vaccine-like action inducing an anti-tumor adaptive immune response. These results represent the both the first reported application of bacterial tumor therapy to a benign neoplasm, as well as, the first demonstration of a schwannoma immunotherapy.

Thus provided herein are methods for a treating a subject having or at risk of having a benign nervous system tumor. The methods include administering to the subject a therapeutically effective amount of a composition comprising live attenuated Salmonella bacteria, optionally in combination with an immune checkpoint inhibitor and/or angiogenesis inhibitor. Also provided herein are compositions comprising live attenuated Salmonella bacteria, optionally in combination with a checkpoint inhibitor and/or angiogenesis inhibitor, for use in a method of a treating a subject having or at risk of having a benign nervous system tumor.

In some embodiments, the subject is a subject having or diagnosed as having a benign tumor or tumor-associated condition selected from the group consisting of: neurofibromatosis 1 (NF1); neurofibromatosis 2 (NF2); schwannomatosis; meningioma; schwannoma; vestibular schwannoma; sporadic schwannoma; neurofibroma; neurofibromatosis (NF); or any combination thereof. In some embodiments, the subject does not have a malignant solid tumor (i.e., has not been diagnosed with a malignant solid tumor). In some embodiments, the subject has a condition associated with an increased risk of a benign nervous system tumor, e.g., neurofibromatosis 1 (NF1); neurofibromatosis 2 (NF2); or schwannomatosis.

In some embodiments, the attenuated Salmonella is administered intratumorally or intravenously.

In some embodiments, the attenuated Salmonella is an attenuated strain of S. typhimurium, e.g., Salmonella enterica serovar typhimurium strain VNP20009 with modified lipid A (msbB−) and purine auxotrophic mutation (purI−).

In some embodiments, the composition does not comprise Clostridium novyi.

In some embodiments, the attenuated Salmonella do not comprise a lysis gene or cassette operably linked to an intracellularly induced Salmonella promoter.

In some embodiments, the checkpoint inhibitor is an inhibitor of PD-1 or CTLA-4 signaling, e.g., an antibody that binds to PD-1, CD40, PD-L1, or CTLA-4.

In some embodiments, the angiogenesis inhibitor is an inhibitor of vascular endothelial growth factor (VEGF) or its receptor (VEGFR), e.g., Bevacizumab.

Further, provided herein are methods to treat benign nerve sheath tumors in a mammal comprised of administering to said mammal a therapeutically effective dose or titer of an attenuated strain of pathogenic enteric bacteria. In some embodiments, the attenuated strain of pathogenic enteric bacteria is Salmonella typhimurium. In some embodiments, the attenuated strain of Salmonella typhimurium has purI and msbB gene deletions, said strain named VNP20009. In some embodiments, the attenuated strain of Salmonella typhimurium is defective in guanosine 5′diphosphate-3′-diphosphate synthesis, said strain named 8ppGpp. In some embodiments, the administration includes, but is not limited, intravenous injection or by way of direct injection into the benign nerve sheath tumor. In some embodiments, the nerve sheath tumor includes, but is not limited to, a neurofibroma or schwannoma. In some embodiments, the tumor includes, but is not limited to those associated with Neurofibromatosis type 1, Neurofibromatosis type 2, Schwannomatosis, or sporadic schwannoma.

In some embodiments, the methods include administering to said mammal therapeutically effective doses of an attenuated strain of pathogenic enteric bacteria and a checkpoint inhibitor. In some embodiments, the checkpoint inhibitor, includes, but is not limited to, a peptide, antibody, small molecule, microRNA, antisense oligonucleotide, or small interfering RNA. In some embodiments, the checkpoint inhibitor is a monoclonal antibody that binds to the epitope of an antigen. In some embodiments, the monoclonal antibody-binding epitope is in a PD-1 or CTLA-4 antigen.

In some embodiments, the mammal is a human.

Also provided herein are pharmaceutical compositions comprised of an attenuated strain of pathogenic enteric bacteria in a pharmaceutically acceptable carrier, and optionally a checkpoint inhibitor. In some embodiments, the attenuated strain of pathogenic enteric bacteria is Salmonella typhimurium.

In some embodiments, the checkpoint inhibitor is a monoclonal antibody. In some embodiments, the monoclonal antibody binds to an epitope of PD-1 or CTLA-4 antigen.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIG.s, and from the claims.

### DESCRIPTION OF THE FIGURES

FIGS. 1A-D. Intratumoral injection of attenuated S. typhimurium controls schwannoma development in human HEI-193 xenograft and mouse 08031-9 allograft schwannoma models. A) VNP20009 and ΔppGpp caused significant tumor regression following i.t. injection 2-weeks post tumor cell implantation (n=8 mice/group). B) VNP20009, not ΔppGpp, injection 1-week after tumor cell implantation led to growth control (n=8 mice/group). I.t. VNP20009 and ΔppGpp injection led to increased apoptosis in both the HEI-193 xenograft (C) and 08031-9 allograft models (D) compared to PBS injected controls (n=3 mice/group, yellow arrow heads indicate representative apoptotic bodies). Repeated measures ANOVA was used to compare tumor signals between groups, and one-way ANOVA used for apoptotic body analysis. Data are shown as mean±SEM. *p<0.05, **p<0.01. ***p<0.001.

FIGS. 2A-F. S. typhimurium injection of intrasciatic allograft schwannomas increased pro-inflammatory cytokines and altered immune cell infiltration. (A) S. typhimurium injected nerves of the mouse allograft schwannoma model showed an increased immune cell infiltration (CD45+ common leukocytes and CD68+ pan macrophages) in comparison to PBS injected tumors, (yellow arrowheads) indicate the positive staining (n=3 mice/group). (B) Quantification of the CD45+ cells (left) and CD68+ cells (right) showed a significant elevation in the leukocytes and macrophages in the tumors of the VNP20009 and ΔppGpp injected mice, compared to the PBS control. (C) Flowcytometric analysis of the tumor associated macrophages which were identified as CD45+F4/80+ subset. CD86 expression was used to identify M1 macrophages while CD206 expression was used to identify M2 macrophages. The ratios of M1 to M2 (M1/M2) were calculated as % M1 (CD68+) population in CD45+F4/80+ divided by % M2 (CD206+) population in CD45++F4/80+. M1/M2 ratios of TAMs on days 3 (left) and 7 (right). (D) Adaptive immune cells infiltration of the injected tumors was analyzed by flow cytometry at 7-days post bacterial injection. The indicated percentages represent CD4+ T cells (CD3+CD4+), CD8+ T cells (CD3+CD8+), and CD+25 T-cells (CD4+CD25+). Flow cytometric profiles are presented in FIG. 10. Quantitative RT-PCR (E) and ELISA (F) of cytokines and inflammasomes in tumor microenvironment 3-days post S. typhimurium injection. One-way ANOVA was used to compare between the different treatments. Data are shown as mean±SEM. N=3/group. Asterisk (*) denotes difference compared to PBS control; hash sign (#) denotes difference compared to ΔppGpp. */# p<0.05, **/## p<0.01. ***/### p<0.001.

FIGS. 3A-E. I.t. VNP20009 injection inhibits tumor growth of primary-treated and uninjected distal schwannomas, and addition of systemic anti-PD-1 mAb enhances killing of the primary tumor. A) Experimental protocol of the combination therapy with VNP20009 and anti-PD-1 mAb. B) 08031-9 cells were implanted subcutaneously on both flanks of FVB/n mice (n=6 mice/group). Significant tumor regression was observed in the mice tumors injected with monotherapy of anti-PD-1 mAb or VNP20009, in comparison to PBS. Combination therapy regressed tumor more significantly than monotherapy of either VNP20009 or anti-PD-1 mAb. C) The injected tumor sites were harvested at the end of the study and analyzed by flowcytometry for cytotoxic CD8+, Helper CD4+ and regulatory CD25+ T cells (N=3/group). D) While anti-PD-1 mAb, significantly reduced tumor growth in the un-injected site compared to PBS, VNP20009 monotherapy and VNP20009/anti-PD-1 mAb combination significantly enhanced the tumor growth control in the un-injected site compared to the anti-PD-1 mAb or PBS treated group. E) the un-injected tumor sites were harvested on day 26 post-implantation and analyzed by flowcytometry for cytotoxic CD8+, Helper CD4+ and regulatory CD25+ T cells (N=3/group). Flow cytometric profiles are presented in FIG. 9. Repeated measure ANOVA was utilized to compare the tumor volumes and/or signals between the different groups. One-way ANOVA was used to compare the flowcytometric data between the different groups. Data are shown as mean±SEM. *p<0.05, **p<0.01.

FIGS. 4A-E. I.t. VNP20009 injection inhibits tumor growth of the injected and subsequently implanted experimental schwannomas, and addition of systemic anti-PD-1 mAb enhances killing of the injected tumor. A) Experimental protocol of the combination therapy with VNP20009 and anti-PD-1 mAb. B) 08031-9 cells were implanted subcutaneously in the left flank of FVB/n mice (n=6/group). Significant tumor regression was observed in the mice tumors injected with monotherapy of anti-PD-1 mAb or VNP20009, in comparison to PBS. Combination therapy regressed tumor more significantly than monotherapy of either VNP20009 or anti-PD-1mAb. C) The injected tumor sites were harvested at the end of the study and analyzed by flowcytometry for cytotoxic CD8+, Helper CD4+ and regulatory CD25+ T cells. D) 08031-9FC tumor cells expressing firefly luciferase were implanted in the distal sciatic nerve of the primarily injected mice on day 20 post-implantation (n=5/group), tumor was monitored by bioluminescence. E) the secondary tumor sites were harvested 16-days post implantation and analyzed by flowcytometry for cytotoxic CD8+, Helper CD4+ and regulatory CD25+ T cells. Flow cytometric profiles are presented in FIG. 10. Repeated measures ANOVA was utilized for analysis of tumor volume and bioluminescence signals. One-way ANOVA was used for analysis of flowcytometric data. Data are shown as mean±SEM. *p<0.05, **p<0.01.

FIG. 5. Intratumoral S. typhimurium injection inhibits angiogenesis in intrasciatic allograft murine-schwannomas. VNP20009- and ΔppGpp-injected tumors harvested 2-weeks following bacterial injection showed decreased vascularization compared to PBS controls as determined by CD31+ staining, as well as, by direct visualization. Immunohistochemistry is based on 3 tumors/group, representative staining is shown, and red arrowheads indicate positive endothelial cells. CD31+ cellular profiles were quantified using Image-J and one-way ANOVA was used for data analysis. Data are shown as mean±SEM. ***p<0.001.

FIGS. 6A-D. I.t. S. typhimurium injection suppresses growth of xenograft human-NF1, human sporadic MPNST, and human-meningioma subcutaneous tumors. Intratumoral injection of either VNP20009 or ΔppGpp caused regression of NF-1 associated (S462TY, A) and growth control of sporadic (STS26T, B) malignant peripheral nerve sheath tumors (MPNST), compared to PBS injected tumors. Similarly, i.t. injection of VNP20009 or ΔppGpp led to regression of benign Ben-Men-1 meningioma (C) and growth control of malignant meningioma CH-157 (D) tumor growth, compared to PBS injected tumors. Arrows indicate the time of bacterial/PBS injection. Repeated measures ANOVA was used to compare tumor size between groups. N=5 mice/group. Data are shown as mean±SEM. ***p<0.001 (PBS compared to either Salmonella strain).

FIGS. 7A-B. Intratumoral injection of attenuated S. typhimurium (VNP20009) controls schwannoma development in human HEI-193 xenograft and mouse 08031-9 allograft schwannoma models. A) VNP20009 caused significant tumor regression in xenograft schwannoma model following intratumoral (i.t.) injection 2-weeks post tumor cell implantation (n=8 mice/group). B) VNP20009 injection of allograft schwannoma model 1-week after tumor cell implantation led to growth control (n=8 mice/group). Repeated measures ANOVA was used to compare tumor signals between groups. Data are shown as mean±SEM. *p<0.05, **p<0.01. ***p<0.001.

FIG. 8. Salmonella typhimurium injected schwannoma-bearing nerves showed increased altered immune cell infiltration. S. typhimurium injected nerves of the human xenograft model showed an increased immune cell infiltration (CD45+ common leukocytes and CD68+ pan macrophages) in comparison to PBS injected tumors, (yellow arrowheads) indicate the positive staining (n=3 mice/group).

FIG. 9. Flowcytometric analysis of the spleen macrophages in the mice injected with S. typhimurium or PBS. The bacterial injected immunocompetent schwannoma mice showed an increase in the macrophage's population in the spleen of the injected mice 3 days post injection. Flowcytometric analysis of CD45+F4/80+ macrophages showed an in increase in the spleen of the mice injected with VNP20009 (38.8%), ΔppGpp (32.4%), compared to PBS (15.4%).

FIGS. 10A-C. Flowcytometric analysis of the tumor-infiltrating immune cells in the tumors of mice injected with S. typhimurium or PBS. Analysis of M-1 type macrophages (F4/80+CD86+) and M-2 type macrophages (F4/80+CD206+) in the tumors harvested 3 days (A) or 7 days (B) post bacterial or PBS injection. (C) CD4+ T cells (CD3+CD4+), CD8+ T cells (CD3+CD8+), and CD+25 T cells (CD4+CD25+) analysis in the treated mice was conducted 7 days post injection of either bacterial strains or PBS.

FIGS. 11A-B, Flowcytometric profile of the tumor-infiltrating immune cells in the tumors of mice injected with VNP20009, PD-1 mAb, VNP20009/PD-1 mAb or PBS. Analysis of the T-lymphocytes infiltration into the tumor of the injected site (A) and un-injected site (B). Tumors were harvested and CD4+ T cells (CD3+CD4+), CD8+ T cells (CD3+CD8+), and CD+25 T cells (CD4+CD25+) staining was conducted 26 days post injection.

FIGS. 12A-B. Flowcytometric profile of the tumor-infiltrating immune cells in the tumors of mice injected with VNP20009, PD-1 mAb, VNP20009/PD-1 mAb or PBS. Analysis of the T-lymphocytes infiltration into the tumor of the injected site (A) and secondary tumor site (B). Tumors were harvested and CD4+ T cells (CD3+CD4+), CD8+ T cells (CD3+CD8+), and CD+25 T cells (CD4+CD25+) staining was conducted 20 days post implantation of the primary tumor site and 16 days post implantation of the secondary tumor site.

FIGS. 13A-B. Invasiveness assay of attenuated S. typhimurium in cultured macrophages and schwannoma cell lines. (A) Representative pictures of the HEI-193 internalized salmonella strains after being streaked on agar plate for 16 hr. (B) quantification of the invasiveness as % of infectivity showed that VNP20009 is more invasive than ΔppGpp but less invasive than wild type S. typhimurium. The resulting invasion efficiency of wild type S. typhimurium in murine macrophages, human HEI-193 and mouse 08031-9 was approximately 100%, 96% and 58%, respectively. VNP20009 and ΔppGpp invasion efficiency was markedly lower compared with wild type bacteria. The infectivity percentage for VNP20009 in murine macrophages, human HEI-193 and mouse 08031-9 was approximately 38%, 23% and 15%, respectively while ΔppGpp showed more reduced invasiveness with infectivity percentage of 18% for murine macrophages, 10% for HEI-193 and 5% for 08031-9 cells.

FIGS. 14A-B. ELISA of cytokines following exposure of Cultured human schwannoma (HEI-193, A) or mouse schwannoma (08031-9, B) cell lines to S. typhimurium. Quantification of IL-1β, IL-18 and TNF-α by ELISA, following incubation with VNP20009 or ΔppGpp did not show any difference in released cytokines compared to PBS treated cells. One-way ANOVA was used for inter-group comparison. N=3 independent experiments. Data shown as mean±SEM.

FIGS. 15A-B. ELISA of cytokines following exposure of Cultured human (THP-1 differentiated macrophages, A) or mouse (RAW 264.7 macrophages, B) cell lines to S. typhimurium. Quantification of IL-1β, IL-18 and TNF-α by ELISA, following incubation with VNP20009 or ΔppGpp showed significant difference in released cytokines compared to PBS treated cells. One-way ANOVA was used for inter-group comparison. Data shown as mean±SEM. N=3 independent experiments; * p<0.05; **p<0.01.

FIG. 16. S. typhimurium did not induce cytokines release in serum of mice injected direct into tumors of allograft immune-competent-schwannoma mouse model. ELISA of cytokines in the serum of the injected mice was measured 3 days post bacterial. One-way ANOVA was used for inter-group comparison. Data shown as mean±SEM. N=3 independent experiments.

### DETAILED DESCRIPTION

Bacteria-mediated cancer therapy (BCT) utilizing gram negative organisms was introduced by William Coley in the mid-19th century when he utilized live Streptococcus pyogenes to treat solid tumors¹². The rationale for bacterial cancer therapy is that some bacterial strains, including the gram-negative bacteria Salmonella typhimurium (S. typhimurium)^{13-21,} can specifically home to and proliferate within hypoxic areas of angiogenic tumors inducing both direct lysis of tumor cells, as well as, establishment of anti-tumor immune responses²². Further, bacterial injection of tumors has been shown to be anti-angiogenic^{23,24}. Thus, in addition to directly inducing cancer cell death, bacteria can act as immune-oncology and anti-angiogenic agents targeting highly vascularized tumors and establishing an immune control preventing the development of new tumors.

There is a substantial body of preclinical and clinical data supporting BCT as an immunotherapeutic strategy^{20,25,32,57,58} and for 4 decades intravesical application of a live attenuated strain of Mycobacterium bovis has been the only FDA-approved treatment of bladder carcinoma in situ²⁹. BCT utilizing attenuated strains of S. typhimurium has demonstrated clear efficacy in several preclinical cancer models^{16,18-20} Early-phase clinical testing of attenuated S. typhimurium-based BCT utilizing intravenous, direct intratumoral or oral delivery have demonstrated safety but failed to show efficacy^{25,26,27, 28}. This lack of efficacy may be due to the rapid division of cancer cells, as well as, use of intravenous delivery. Bacterial inoculum is limited by toxicity with systemic delivery and in these trials lack of efficacy may have been dose related. There is currently one BCT approved by the U.S. Food and Drug Administration: a live attenuated strain of Mycobacterium bovis that for the last 4 decades has been the standard of care for high-risk non-muscle-invasive bladder cancer²⁹.

However, BCT has never been suggested as a possibility for benign neoplasms, perhaps because benign tumors tend to be immunologically cold^{66,67}. Thus, bacteria therapy has never been tested in the context of slow-growing benign tumors, such as schwannomas, for which traditional cancer therapies targeting mainly highly replicating cells are not effective. Provided herein is a preclinical study supporting bacterial treatment of schwannoma, a benign neoplasm of the peripheral nervous system. It was hypothesized that intratumoral injection of attenuated S. typhimurium could have the potential to directly kill schwannoma cells, inhibit angiogenesis, and convert the immunologic tumor micro-environment from one that is relatively ‘cold’ to ‘hot.’ It was further hypothesized that the combination of immunologic cell death (were it to occur), generation of a pro-immunogenic tumor environment, and VEGF/angiogenesis inhibition could synergize to generate an adaptive anti-tumor immune response.

To test these hypotheses, the effects of two attenuated S. typhimurium strains (VNP20009 and ΔppGpp) were evaluated in both a xenograft human-NF2 model in nude mice and an allograft mouse-schwannoma model in syngeneic immune competent FVB/N mice. The data showed that intratumoral injection of attenuated S. typhimurium controlled schwannoma growth in both models. I.T. S. typhimurium injection of schwannoma results in tumor cell killing and, in immune competent mice, induction of a systemic anti-tumor adaptive immune response. This anti-tumor immune response controlled growth of non-bacterially-injected tumors that were present at the time of bacterial treatment, as well as preventing development of “rechallenge” tumors following treatment. S. typhimurium increased tumor infiltrating CD4+ helper and CD8+ cytotoxic T cells, and decreased CD25+ Tregs in bacterially-injected and contralateral non-injected contralateral and rechallenge (other than the effect on CD4+ cells) allograft schwannomas, further supporting the presence of an anti-tumor adaptive immune response. Addition of systemic PD-1 immune checkpoint inhibition to i.t. S. typhimurium injection enhanced schwannoma control of bacterially-injected and contralateral non-injected, but not rechallenge, tumors. Investigation of tumor infiltrating lymphocytes (TILs) demonstrated increased numbers of CD4+ helper and CD8+ cytotoxic T cells and decreased numbers of CD25+ regulatory T cells in schwannomas injected with attenuated S. typhimurium.

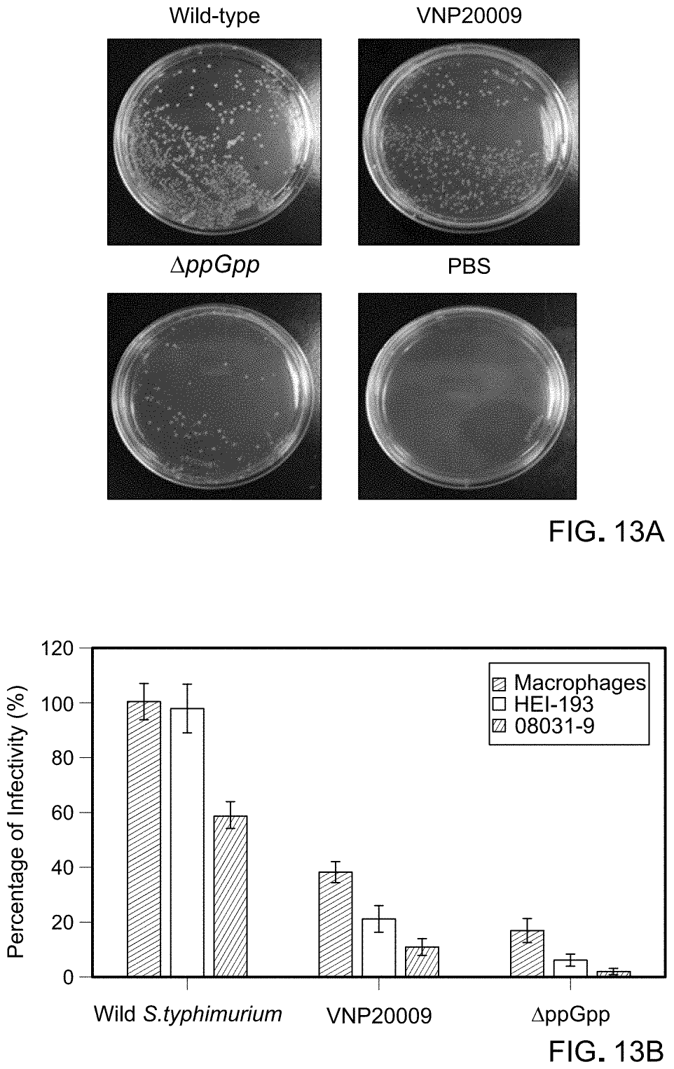

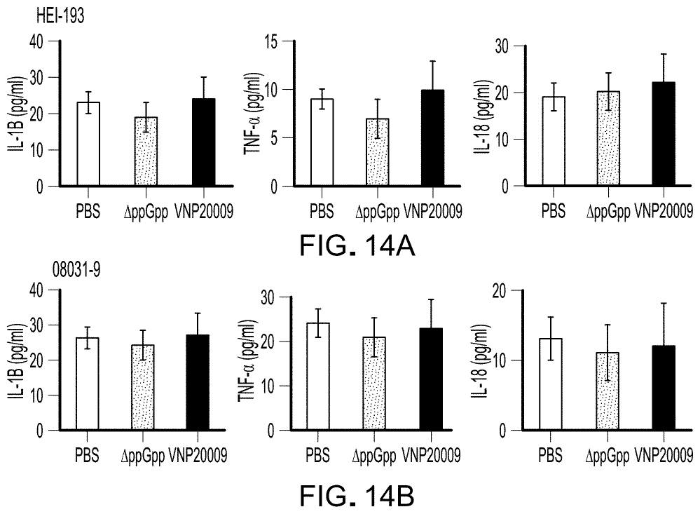

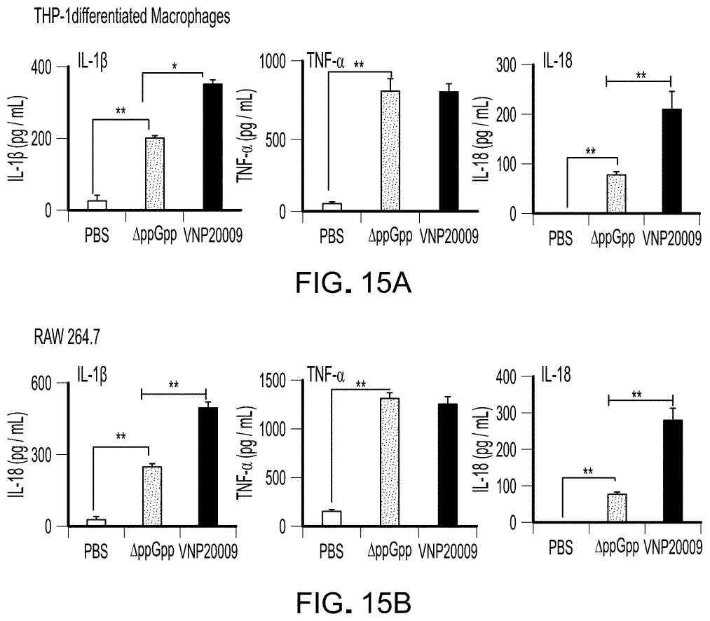

The present study tested the capacity of two attenuated S. typhimurium strains, VNP20009 and ΔppGpp, to suppress schwannoma growth. Attenuation confers decreased likelihood of pathogenicity, including septic shock, in both strains. In vitro evaluation revealed that VNP20009 is more invasive than ΔppGpp in both cultured macrophages and schwannoma cell lines, but less invasive than wild type S. typhimurium (FIGS. 13A&B). While exposure of cultured macrophages to both S. typhimurium strains resulted in release of inflammatory cytokines, co-culture of neither VNP20009 nor ΔppGpp with schwannoma cell lines induced any cytokine release (FIGS. 14A&B and 15A&B). This suggests that the bacterial-macrophage interaction may play a critical role in the observed S. typhimurium antitumor effect.

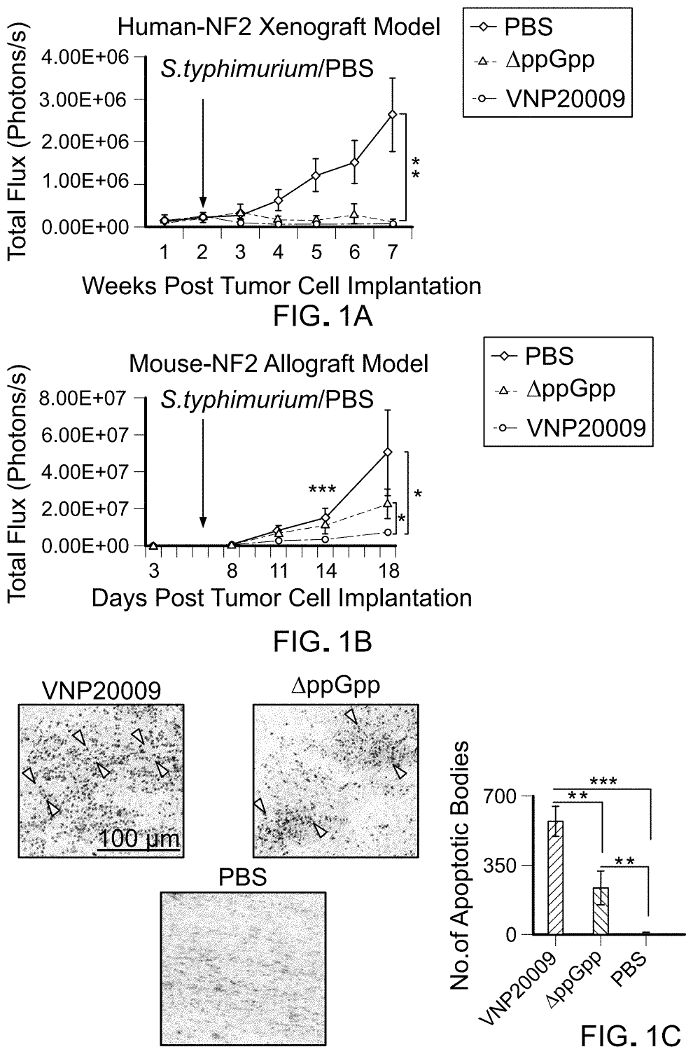

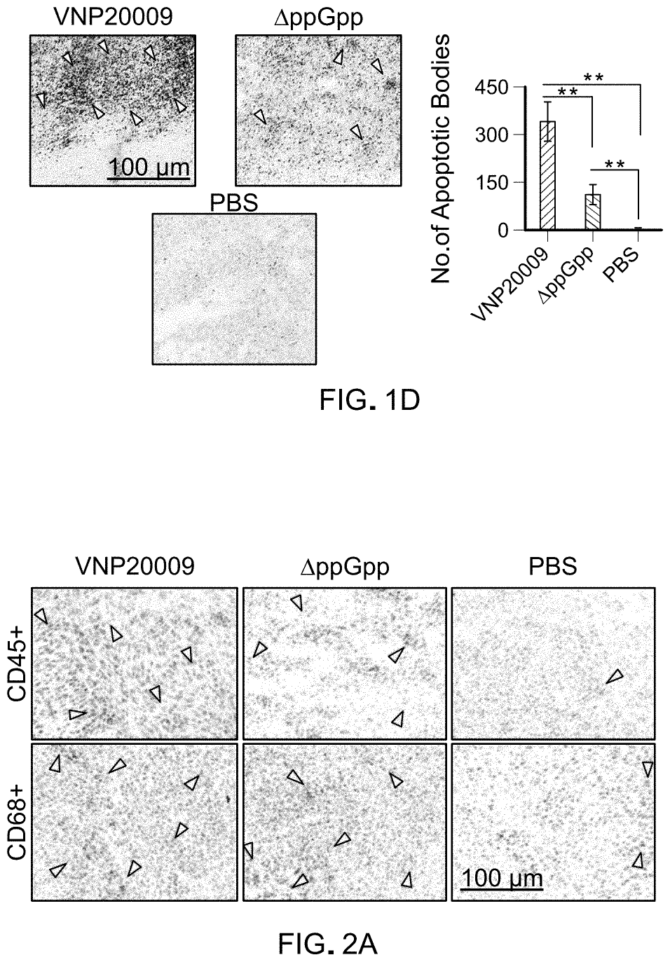

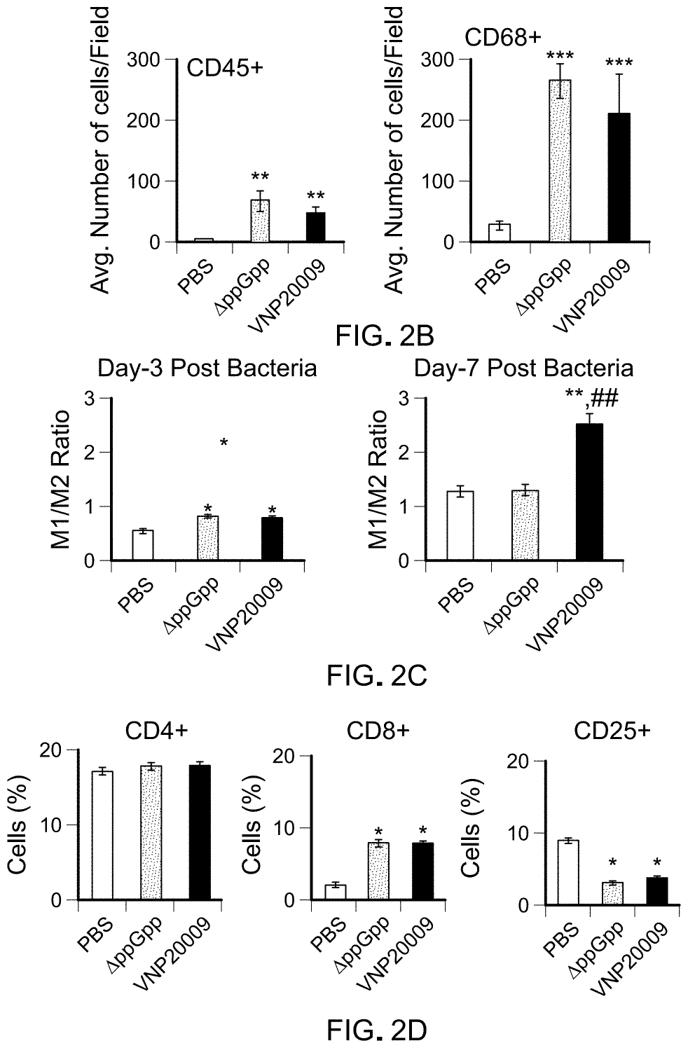

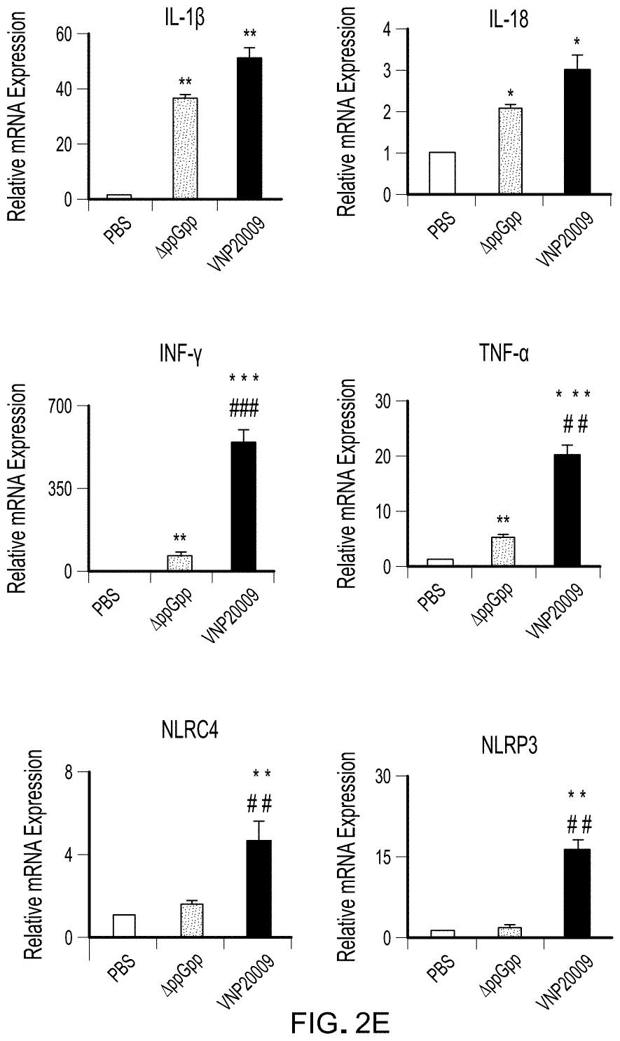

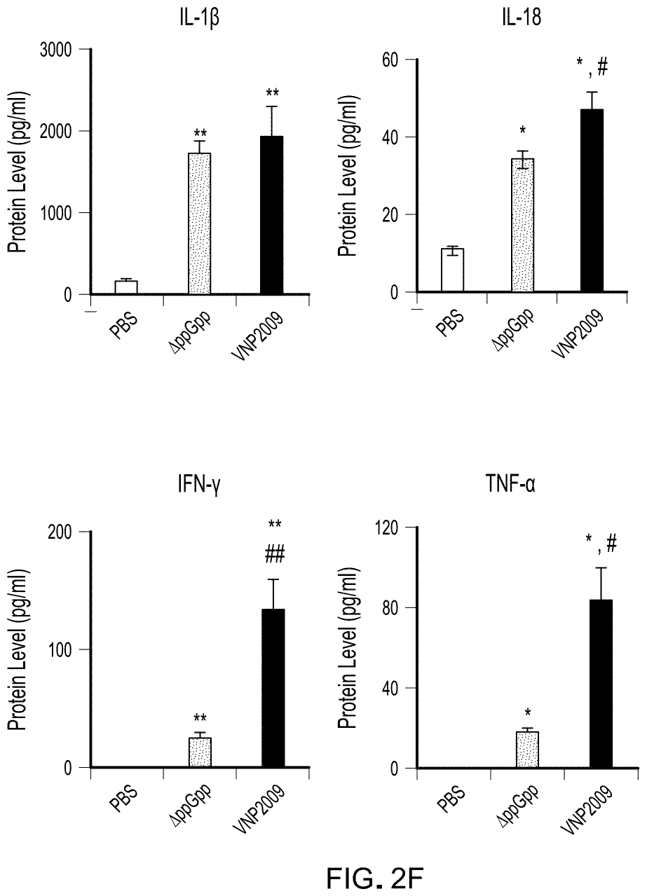

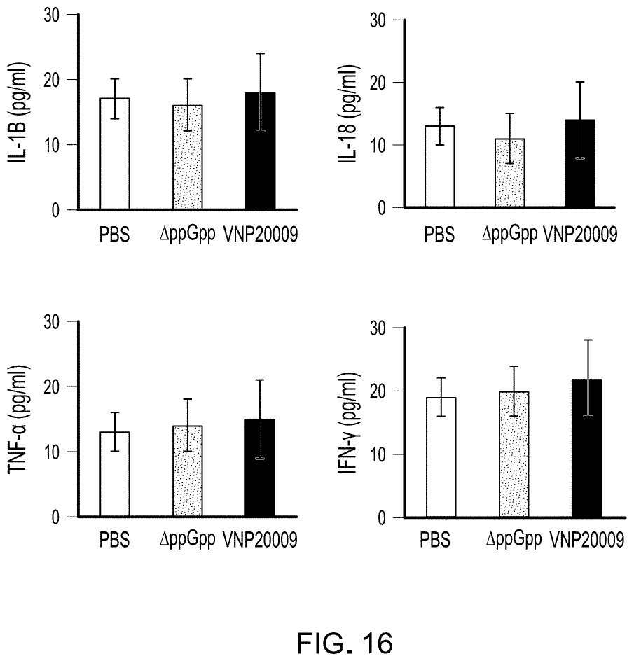

The in vivo data showed that intratumoral (i.t.) injection of either VNP20009 or ΔppGpp monotherapy regressed tumor growth in our human NF2 xenograft model with no difference in the regression magnitude between the two tested strains. Further, in the allograft schwannoma model in immunocompetent mice i.t. injection of VNP20009 controlled tumor growth. The therapeutic efficacy of VNP20009 was mirrored by an increase in the apoptotic bodies (FIG. 1D) and increase in the release of inflammatory cytokines, including IL-18, TNF-α and IFN-γ, in the tumor microenvironment, compared to ΔppGpp or PBS (FIG. 2E&F). No increased systemic cytokine levels were observed in mice injected with S. typhimurium (FIG. 16). Further studies focused on analyzing the immune profile alteration in the tumor microenvironment of the immune competent schwannoma model following bacterial or PBS i.t. injection of VNP20009.

M2-type macrophage and myeloid-derived suppressor cells (MDSC) have been shown to infiltrate vestibular schwannomas and are associated with progressive tumor growth^{42,59} Unlike M2 type macrophages, which are tumor promoting, M1-type macrophages are immunostimulatory and inhibit tumor growth and shape the adaptive immune response at least in part via phagocytosis and antigen presentation⁶⁰⁻⁶⁵. In the allograft schwannoma model, i.t. VNP20009 injection which controlled tumor growth increased the ration of M1 to M2 macrophages among CD45+F4/80+ tumor activated macrophages (TAMs) by day-3 following bacterial injection.

Methods of Treatment

As shown herein, VNP20009, a strain of attenuated Salmonella, e.g., S. typhimurium, that has been safely administered to patients with metastatic melanoma and renal cell carcinoma^{25,30,31}, was effective in treating schwannoma models in mice. Schwannomas are genetically stable, slow growing, and highly vascularized with large hypoxic areas. These features could make schwannomas an ideal homing environment for bacteria and a potentially perfect target for bacteria cytotoxic and anti-angiogenic features. In addition, the capacity of bacteria to induce immune responses allows treatment of multiple distal lesions and the establishment of control mechanisms that prevent the occurrence of new schwannomas throughout a patient's life, a feature typical of these tumors.

The methods described herein include methods for the treatment of benign nervous system tumors. In some embodiments, the tumor is a schwannoma. Schwannoma tumors are composed of Schwann-lineage cells and form along peripheral, spinal and cranial nerves. These tumors can cause pain, sensory/motor dysfunction, and death through compression of peripheral nerves, the spinal cord, and/or the brain stem. Multiple schwannomas in peripheral distal and intracranial nerves are the hallmark of neurofibromatosis 1 and 2 (NF1 and NF2), and schwannomatosis, three types of nerve sheath tumors. Schwannomas are benign tumors composed of neoplastic dedifferentiated Schwann cells. Although typically nonmalignant and slow growing, these tumors can have devastating consequences for patients. They can cause extreme pain and compromise sensory/motor functions, including hearing and vision. Schwannomas in NF2 are frequently associated with neurological deficits, such as paresthesias, weakness, or hearing loss, and similar tumors in schwannomatosis often cause excruciating pain. Some schwannomas become very large, causing compression of adjacent organs or structures, and can lead to paralysis or death due to progressive spinal cord or brainstem compression. Schwannomas may arise sporadically, without presenting any genetic features of NF1, NF2 and schwannomatosis. Most vestibular schwannomas are sporadic schwannomas, so their incidence is very significant. Vestibular schwannomas usually occur as single tumors, not as multiple tumors throughout the body. In some embodiments of any of the aspects, a subject in need of treatment for a schwannoma can be a subject having or diagnosed as having a condition selected from the group consisting of: neurofibromatosis 1 (NF1); neurofibromatosis 2 (NF2); schwannomatosis; meningioma; nerve sheath tumor; schwannoma; vestibular schwannoma; sporadic schwannoma; neurofibrosarcoma; neurofibroma; neurofibromatosis (NF); malignant peripheral nerve sheath tumor; and a combination thereof. Subjects who can be treated using the present methods include mammals, e.g., humans and non-human veterinary subjects, e.g., cats, dogs, horses, goats, cows, and so on.

The present standard of care for patients with NF2 and schwannomatosis is surgical resection or radiosurgery of symptomatic tumors to reduce tumor size. Unlike in the case of sporadic schwannomas, in which typically only a single tumor is present and surgery is generally an efficacious treatment strategy as long as the lesion is accessible for resection, in schwannomatosis and NF2, which present with multiple tumors, resection is confounded by both the inaccessibility of many tumors and by risk of nerve damage, including major motor dysfunction, significant sensory loss (including deafness in the case of NF2 vestibular schwannomas), and neuropathic pain. Thus, for most individuals there is substantial morbidity associated with schwannomas in both NF2 and schwannomatosis, as well as with the current therapies. This suffering and debility, in combination with the paucity of therapeutic options, makes the treatment of schwannomas a major unmet medical need.

Generally, the methods include administering a therapeutically effective amount of attenuated Salmonella, e.g., S. typhimurium as described herein, optionally in combination with a checkpoint inhibitor, to a subject who is in need of, or who has been determined to be in need of, such treatment. Examples of routes of administration include parenteral, e.g., intravenous, intradermal, subcutaneous, and intratumoral (i.t.) administration. In preferred embodiments, the i.t. route is used to maximize bacterial dose and minimize potential dose limiting toxicity (DLT). One of skill in the art would be able to identify a subject as having a benign nervous system tumor. In some embodiments, the subject is a subject having or diagnosed as having a benign tumor or tumor-associated condition selected from the group consisting of: neurofibromatosis 1 (NF1); neurofibromatosis 2 (NF2); schwannomatosis; meningioma; schwannoma; vestibular schwannoma; sporadic schwannoma; neurofibroma; neurofibromatosis (NF); or any combination thereof. In some embodiments, the subject does not have a malignant solid tumor, e.g., does not have cancer. In some embodiments, the subject has a condition associated with an increased risk of a benign nervous system tumor, e.g., neurofibromatosis 1 (NF1); neurofibromatosis 2 (NF2); or schwannomatosis.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular anti-tumor effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation. Administration of a therapeutically effective amount of a compound described herein for the treatment of a benign nervous system tumors can result in decreased tumor size, tumor number, tumor growth rate, or likelihood of recurrence, e.g., after treatment with a method described herein.

The present methods thus include the administration of attenuated S. typhimurium strains to suppress tumor growth. As shown herein, to enhance efficacy of the therapy, in preferred embodiments the present methods can utilize intra-tumoral injection of bacteria, rather than intravenous delivery, which increases bacterial concentration within tumor and minimizing systemic toxicity. As shown herein, direct injection of attenuated S. typhimurium into schwannoma had a vaccine-like action inducing an anti-tumor adaptive immune response.

Attenuated S. typhimurium

As used herein, the term “attenuated” refers to a strain that has been rendered to be less virulent compared to the native strain, thus becoming harmless or less virulent. Attenuated does not mean inactivated. Attenuation confers decreased likelihood of pathogenicity, including septic shock, in both strains. Although the present data relates primarily to VNP20009 and ΔppGpp, other attenuated strains can also be used. Methods of generating attenuated Salmonella strains are known in the art, including directed or random mutagenesis followed by screening for reduced virulence. Directed mutation, e.g., of the aroA gene (aroA is part of the shikimate pathway connecting glycolysis to synthesis of aromatic amino acids; aroA deficient Salmonella strains are described e.g. in Feigner et al, mBio, 2016, 7: e01220-16); the gene purI (defective in purine synthesis); or the asd gene (defective in aspartate-semialdehyde dehydrogenase required for cell wall synthesis) can be used. Attenuated strains of salmonella are disclosed in WO 2014/005683; WO 2016/202459; WO 2013/09189; and US 20200038496 (attenuated S. typhi Ty21a). Strains that can be used in the present methods include attenuated versions of Salmonella enterica serovar typhimurium (“S. typhimurium”), Salmonella montevideo, Salmonella enterica serovar Typhi (“S. typhi”), Salmonella enterica serovar Paratyphi B (“S. paratyphi B”), Salmonella enterica serovar Paratyphi C (“S. paratyphi C”), Salmonella enterica serovar Hadar (“S. hadar”), Salmonella enterica serovar Enteriditis (“S. enteriditis”), Salmonella enterica serovar Kentucky (“S. kentucky”), Salmonella enterica serovar Infantis (“S. infantis”), Salmonella enterica serovar Pullorurn (“S. pullorum”), Salmonella enterica serovar Gallinarum (“S. gallinarum”), Salmonella enterica serovar Muenchen (“S. muenchen”), Salmonella enterica serovar Anaturn (“S. anatum”), Salmonella enterica serovar Dublin (“S. dublin”), Salmonella enterica serovar Derby (“S. derby”), Salmonella enterica serovar Choleraesuis var. kunzendorf (“S. cholerae kunzendorf”), and Salmonella enterica serovar minnesota (“S. minnesota”). See, e.g., WO/2008/039408 and US 20200023053; US 20190153452; US 20170333490 and US 20180339032; Grant et al., PLoS Pathog. 2012 December; 8(12): e1003070; Tennant and Levine, Vaccine. 2015 Jun. 19; 33(0 3): C36-C41.

In preferred embodiments, the attenuated strains used in the present methods do not comprise Clostridium novyi (see, e.g., WO2014160950). In preferred embodiments, the attenuated strains used in the present methods do not comprise a lysis gene or cassette operably linked to an intracellularly induced Salmonella promoter (see, e.g., US 20170333490).

Combination Therapy

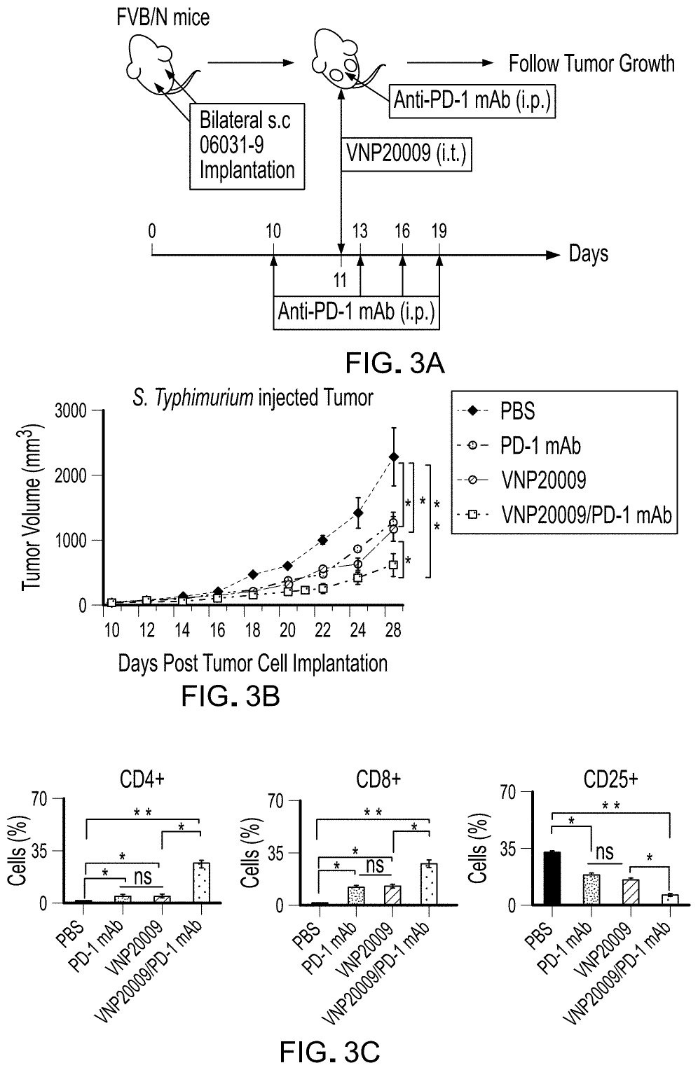

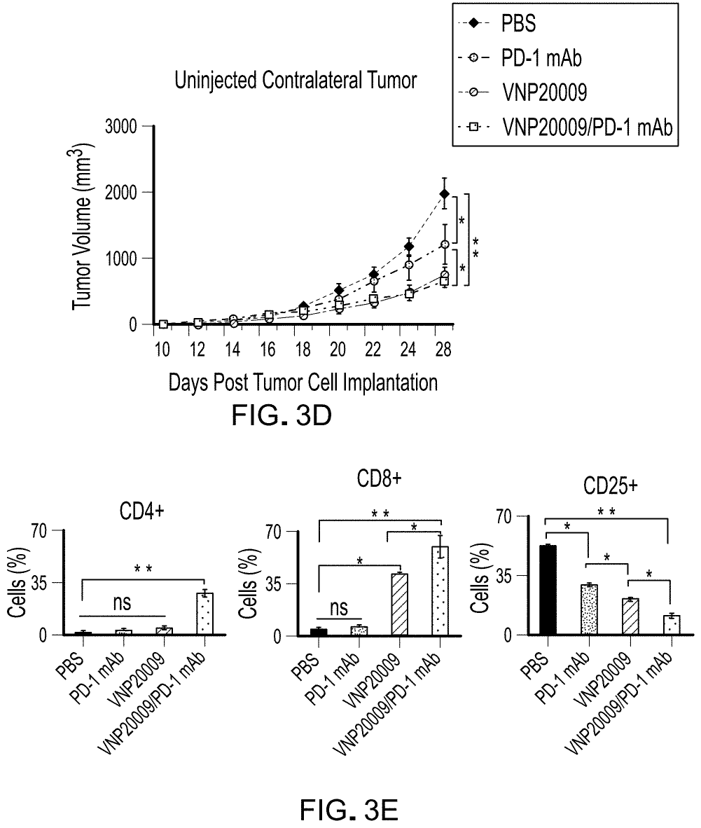

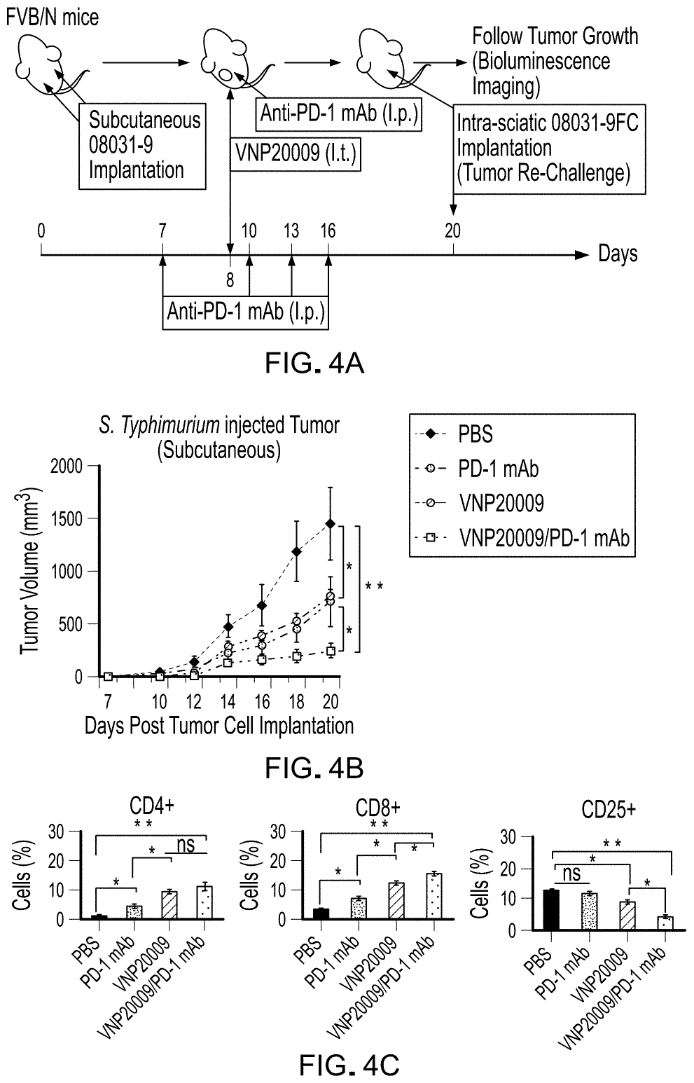

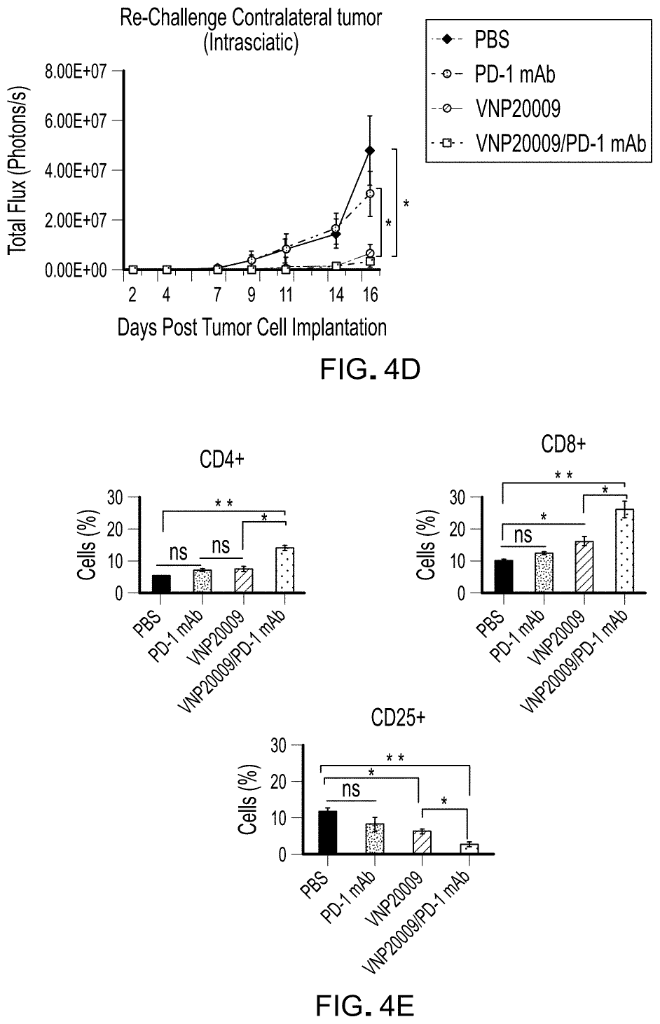

The present methods can include administration of the attenuated Salmonella strain in combination one or more other treatments. For example, the present studies demonstrated that i.t. VNP20009 of schwannoma in immunocompetent mice resulted in an increase in the percentage of tumoral helper CD4+ and cytotoxic CD8+ T cells, and concomitant decrease in percentage of CD+25 Tregs. These changes in tumor infiltrating T cell populations in conjunction with a shift to M1 tumoricidal macrophages is suggestive of S. typhimurium induced adaptive anti-tumor immune response. The high PD-L1 expression reported in schwannomas indicates resistance to cell-mediated immunity in the tumor immune microenvironment⁴⁹. Given this, the effect of addition of PD-1 immune checkpoint inhibition on efficacy of i.t. S. typhimurium (VNP20009)-associated schwannoma growth control and development of host anti-tumor adaptive immunity was evaluated. The data showed that this combination resulted in enhanced tumor regression of bacterially injected schwannomas that was associated with an elevation in numbers of CD4+ helper and CD8+ cytotoxic T cells, a reduction in number of CD25+ regulatory T cells infiltrating both bacterially-injected and non-injected tumors (FIGS. 3 and 4). While in contralateral non-bacterially-injected schwannomas (FIG. 3 D,E) and in the rechallenge schwannomas (FIG. 4 D,E) the combination of VNP20009 and anti-PD-1 mAb led to the same enhanced effects on the T cell populations (increased CD4+ and CD8+, and decreased CD25+) compared to bacterially-injected tumors, there was no difference in growth suppression between VNP20009/anti-PD-1 mAb and VNP20009 alone. On the other hand, the effect of both i.t. VNP20009 and the VNP20009/anti-PD-1 mAb combination on schwannoma growth control appears to be greater in the rechallenge tumors (FIG. 4D) than in the primary, bacterially-injected tumors (FIG. 4B). The differences in biology of the bacterially-injected and the non-injected contralateral and rechallenge schwannomas that explain these observations remains to be elucidated.

Thus, the present methods can include administering (together or separately) a combination of bacteria together with a checkpoint inhibitor, e.g., an inhibitor of PD-1 signaling, e.g., an antibody that binds to PD-1, CD40, or PD-L1, or an inhibitor of Tim3 or Lag3, e.g., an antibody that binds to Tim3 or Lag3, or an antibody that binds to CTLA-4.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; and U.S. Pat. No. 8,591,900, including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3:15-25 (2010); Storz, MAbs. 2016 January; 8(1): 10-26; US2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab, Tremelimumab, and EPR1476.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KNO35, Atezolizumab (Tecentriq, MPDL3280A), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP_116171.3. Exemplary antibodies are described in WO2016071448; U.S. Pat. No. 8,552,156; and US PGPub. Nos. 20180298097; 20180251549; 20180230431; 20180072804; 20180016336; 20170313783; 20170114135; 20160257758; 20160257749; 20150086574; and 20130022623, and include LY3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 March; 276(1):80-96; Antoni et al., Am Soc Clin Oncol Educ Book. 2016; 35:e450-8; US PGPub. Nos. 20180326054; 20180251767; 20180230431; 20170334995; 20170290914; 20170101472; 20170022273; 20160303124, and include BMS-986016.

The present methods can also include administering (together or separately) a combination of bacteria together with an angiogenesis inhibitor. A number of angiogenesis inhibitors are known, including those that target vascular endothelial growth factor (VEGF), its receptor (VEGFR), or other molecules involved in angiogenesis. Specific examples include Axitinib (INLYTA); Bevacizumab (AVASTIN); Cabozantinib (COMETRIQ); Everolimus (AFINITOR); Lenalidomide (REVLIIVIID); Lenvatinib mesylate (LENVIMA); Pazopanib (VOTRIENT); Ramucirumab (CYRAIVIZA); Regorafenib (STIVARGA); Sorafenib (NEXAVAR); Sunitinib (SUTENT); Thalidomide (SYNOVIR, THALOMID); Vandetanib (CAPRELSA); or Ziv-aflibercept (ZALTRAP). See, e.g., Zhang et al., Exp Neurol. 2018 January; 299(Pt B):326-333; de Vries et al., Otol Neurotol. 2015 August; 36(7):1128-36; Lim et al., Cancer Treat Rev. 2014 August; 40(7):857-61; Blakeley, Curr Opin Otolaryngol Head Neck Surg. 2012 October; 20(5):372-9; Goel et al., Cold Spring Harb Perspect Med. 2012 March; 2(3):a006486.

Alternatively or in addition, the present methods can be used in combination with surgical resection, e.g., in some embodiment of any of the aspects, the attenuated salmonella strain as described herein can be administered before, concurrently with, or after surgical removal or partial removal of a neoplasm or tumor, e.g., a schwannoma. Various treatment method of the present invention may further comprise treating the subject with surgery, radiation therapy, or chemotherapy, or a combination thereof.

### Pharmaceutical Compositions and Methods of Administration

The methods described herein include the use of pharmaceutical compositions comprising attenuated salmonella as an active ingredient. Pharmaceutical compositions typically include a pharmaceutically acceptable carrier. As used herein the language “pharmaceutically acceptable carrier” includes saline, solvents, dispersion media, and the like, compatible with pharmaceutical administration. Supplementary active compounds can also be incorporated into the compositions, e.g., checkpoint inhibitors and/or angiogenesis inhibitors, e.g., as known in the art and/or discussed herein.

Pharmaceutical compositions are typically formulated to be compatible with its intended route of administration. Examples of routes of administration include parenteral, e.g., intravenous, intradermal, subcutaneous, and intratumoral administration.

### EXAMPLES

The invention is further described in the following examples, which do not limit the scope of the invention described in the claims.

Materials and Methods

The following materials and methods were used in the Examples set forth below.

Cell Culture

The HEI-193 human schwannoma cell line (from D. J. Lim, House Ear Institute, Los Angeles, Calif.) was established from a schwannoma in a patient with NF2, immortalized with human papillomavirus E6/E7 genes and grown as described^{68,69}. Mouse 08031-9 schwannoma cells (from Dr. Marco Giovannini, Univ. of California, Las Angeles, Calif.) were grown as described⁵⁰. The cell lines were infected with lentivirus encoding Fluc (firefly luciferase) and mCherry for bioluminescence imaging and IHC, respectively⁷⁰. Human MPNST (STS.26T) cells were kindly provided by Dr. David Largaespada (Masonic Cancer Center at University of Minnesota), and grown as described⁵⁶. Human NF-1 associated MPNST (S462TY) cells were kindly provided by Dr. Timothy P. Cripe (Center for Childhood Cancer at Nationwide Children's Hospital). Human Ben-Men-1 and CH-157 cells were kindly provided by Dr. Long-Sheng Chang (Center for Childhood Cancer at Nationwide Children's Hospital) and Dr. G. Yancey Gillespie (University of Alabama at Birmingham), respectively. For differentiation of human macrophages, phorbol-12-myristate 13-acetate (PMA) (Sigma-Aldrich, USA) was added to human monocytes in a final concentration of 100 nM. After 24 hours, the PMA supplemented media was removed, cells were washed with PBS and rested in fresh PMA-free media for further 24 hours in order to obtain phenotypic characteristics of macrophages⁷¹. Murine RAW macrophages were obtained (from ATCC, USA). Macrophages were cultured in RPMI media per manufacturer's instructions. All cell lines were confirmed to be free of contamination, including mycoplasma, prior to experimental use.

Bacterial Culture

The attenuated Salmonella enterica serovar typhimurium strain VNP20009 (with modified lipid A (msbB−), purine auxotrophic mutation (purI−)) was purchased (from ATCC, USA, cat #14028) and strain ΔppGpp (with defective ppGpp synthesis (RelA::cat, SpoT::kan)) was kindly provided (Dr. Karsten Tedin, Institut for Microbiology and Epizootics, Centre for Infection Medicine, Berlin, Germany). Bacterial cells were cultured in Luria-Bertani (LB) broth medium with low sodium (DifcoLaboratories, USA) at 37° C. at 300 rpm overnight in aerobic conditions, as previously described^{31,47}. Briefly, Cells were grown to the late-log phase (OD600 nm=0.8) and harvested by centrifugation at 5000 rpm for 10 mins and washed twice with sterile 1× phosphate buffered saline (PBS) before injection into the tumor or infection of cultured cells.

Animals

All animal experiments were approved by and conducted under the oversight of the Massachusetts General Hospital (MGH, Boston, Mass.) Institutional Animal Care and Use Committee (IACUC protocol number 2014N000211). Five-Seven-week-old male mice, nu/nu and FVB/N (Charles River Laboratories) were kept on a 12:12 light-to-dark cycle with ad libitum access to food, water, and daily health checks by Center for Comparative Medicine staff/veterinarian at MGH.

Animal Models and Intratumoral Bacterial Injection

Sciatic nerve schwannomas were generated by direct injection of HEI-193FC human or 08031-8FC mouse schwannoma cells into the left sciatic nerve of isoflurane-anesthetized mice, as described⁷². HEI-193FC or 08031-9FC cells were trypsinized and rinsed with cold PBS, and 30,000 (or 10,000 for 08031-9FC) cells in a volume of 0.5 μl of PBS were injected into the sciatic nerve of athymic nude mice (nu/nu, 5-7-week-old males; National Cancer Institute [NCI]), or syngeneic FVB/N mice (5-7-week-old males; Charles River Laboratories), respectively, using a glass micropipette and a gas-powered microinjector (IM-300; Narishige, Tokyo, Japan). Tumors were injected with 10⁴ CFU Attenuated S. typhimurium (VNP20009 or ΔppGpp) in 2 μl PBS [9, 13] two weeks post HEI-193 tumor-cell implantation or one week post 08031-9 tumor cell implantation, targeting the location of the sciatic nerve where tumor cells were implanted. Tumor growth was monitored by in vivo bioluminescence imaging at weekly intervals for HEI-193 and twice a week for 08031-9, as described⁷². Briefly, mice were injected intraperitoneally with the Fluc substrate d-luciferin, and, 10 min later, signal was acquired with a high efficiency IVIS Spectrum (Caliper Life Sciences, Hopkinton, Mass.). For the immunocompetent subcutaneous model; 08031-9, were resuspended in DMEM and mixed with Matrigel (1:1) (BD bioscience), then grafted subcutaneously (s.c.) in FVB/N syngeneic mice. For the NF-1 or meningioma xenograft models, human S462TY, STS.26T, Ben-Men-1 or CH-157 cells were mixed with Matrigel (1:1) and implanted subcutaneously in nu/nu mice, as described⁵⁶. The tumor volume was estimated using the formula (W×L×L×π/6), where width (W), length (L) are the two largest diameters⁵⁰. When tumor reached 150 mm³, treatment with intraperitoneal (i.p.) PD-1 mAb (250 ug/injection, Bio X Cell, USA) was initiated and repeated every three days, as previously described⁵¹. VNP20009 injection (10⁴ CFU/100 μl PBS) was conducted intratumorally using insulin syringe. Mice in the control group were treated with PBS vehicle or isotype antibody under the same schedule.

Histological and Immunohistochemical Analysis

Animals were terminally anesthetized with isoflurane (3%) and sacrificed by decapitation. Tumor tissues were removed and snap frozen for hematoxylin and eosin (H&E) and immunohistochemical staining, as described⁷³. The tumors were kept in OCT blocks at −80° C. Sections were stained with H&E in accordance with routine protocols. Proliferation marker staining was performed using antibody against Ki67 (Abcam, Cambridge, Mass.). Antibodies against CD45, CD68 and CD31 were utilized for staining of leukocytes, macrophages and vascularization, respectively. All antibodies were purchased from (Abcam, Cambridge, Mass.). Briefly, sections were dried at room temperature (RT) overnight. They were fixed in pre-chilled acetone at 4° C. for 10 min, allowed to dry, and then immediately stained. Sections were washed in PBS, blocked with serum free protein block (Dako, Carpinteria, Calif.) and quenched for peroxidases in dual endogenous enzyme block (Dako). Sections were washed in PBS then incubated with primary antibody for 1 hour at room temperature, then washed in PBS and incubated with horseradish peroxidase-conjugated secondary antibody for 30 min at room temperature (RT). Sections were washed in PBS and incubated with DAB solution (Dako). Counterstaining was accomplished by dipping sections in ethanol and xylene before mounting in Cytoseal (Richard Allan Scientific, San Diego, Calif.) and covered with cover slips for microscopic visualization. In vivo Apoptosis staining was assessed using the TACS 2 TdT-DAB in situ apoptosis Detection Kit (Trevigen, Gaithersburg, Md.). 15 nm sections (cryostat) of fresh frozen nerves fixed with 3.7% formaldehyde after mounting on slides was stained and visualized with diaminobenzidine (DAB) under light microscope per manufacturer's instructions. The utilized antibodies are supplied in Table 1.

TABLE 1
Antibodies used in the study

| | | Company/ | |
|---|---|---|---|
| | Antibody | Catalogue | |
| Antibody name | description | number | Remark |
| Rabbit polyclonal | Rabbit anti-Ki67 | Abcam/ | Primary |
| antibody to Ki67 | | ab15580 | Ab |
| Rabbit polyclonal | Rabbit anti-CD45 | Abcam/ | |
| antibody to CD45 | | ab10558 | |
| Rabbit polyclonal | Rabbit anti-CD68 | Abcam/ | |
| antibody to CD68 | | ab125212 | |
| Rabbit polyclonal | Rabbit anti-CD31 | Abcam/ | |
| antibody to CD31 | | ab28364 | |
| Horseradish | Goat anti-rabbit | Abcam/ | Secondary |
| conjugated Goat | | ab6721 | Ab |
| Anti-Rabbit IgG | | | |
| H&L (HRP) | | | |
| PE-Texas red | MF48017 | Invitrogen | FACS Ab |
| anti-mouse F4/80 | | | |
| APC anti-mouse | mAb (clone | BioLegends | |
| CD206 | 141708) | | |
| APC-CY ™7 | mAb (clone | BD | |
| anti-mouse CD45 | 557659) | Bioscience | |
| PE- anti mouse | mAb (clone | BioLegends | |
| CD86 | 105007) | | |
| APC anti-mouse | Clone 17A2 | BioLegends | |
| CD3 | | | |
| FITC anti-mouse | Clone RM4-5 | BioLegends | |
| CD4 | | | |
| FITC anti-mouse | Clone 53-6.7 | BioLegends | |
| CD8a | | | |
| PE anti-mouse | Clone PC61 | BioLegends | |
| CD25 | | | |
| PE CY ™7 anti- | Clone PK136 | BD | |
| mouse NK1.1 | | Biosciences | |
| APC anti-mouse | I. Clone RB6- | BD | |
| LY6G/LY6C | 8C5 | Biosciences | |

Measurement of Cytokines RNA in Tumors Using Real-Time Quantitative (qRT-PCR)

Tumor tissues was excised at day-3 post bacterial injection, and RNA was extracted by Trizol. Total RNA was transcribed into cDNA using SuperScript™ IV VILO™ with ezDNase enzyme (Invitrogen). The samples were then incubated at 37° C. for 10 min to digest DNA, followed by 25° C. for 10 min, 50° and 85° C. for 5 min in (ProFlex PCR system, Applied Biosystems, USA). For qPCR reactions, 20 ng of the cDNA/well was used as input to determine the target gene expression using Taq-man probes. qPCR assays were performed using the standard cycling mode conditions on a Mx3000P qPCR System (Agilent Technologies, USA). Melt curve analysis was performed using MxPro qPCR software to verify the primer efficiency on each plate and to rule out any non-specific amplifications. The difference in cycle threshold (Ct) values between genes of interest and reference gene 18S (ΔCt) was translated into relative expression using 2-ΔΔCT method and foldchange was calculated by comparing the samples. All reactions were run in triplicate.

Measurement of Cytokines Proteins in Tumors Using ELISA

Tumor tissues were excised at day 3 post bacterial injection, and homogenized in NP40 lysis buffer containing proteinase inhibitors, and the supernatant was collected by centrifugation at 13,000 rpm for 10 mins. Cytokine levels were measured using individual Quantikine ELISA kits (R&D systems, Minneapolis) for human and mouse: Interferon-gamma (IFN-γ) (BD bioscience), TNF-α (BD bioscience), IL-1β/IL-1F2 (BD bioscience) and IL-18 (BD bioscience) according to the manufacturer's instructions. The substrate color reaction was measured at 450 nm with the correction wavelength set at 540 nm or 570 nm using a microplate reader (SpectraMax, Molecular Devices) followed by quantifying the results by standard curves.

In Vitro Invasiveness Assay

Macrophages (human THP-1 and murine RAW 264.7 macrophages) and schwannoma (human HEI-193 and murine 08031-9) cells were grown in 24-well tissue culture plates to a density of 10⁴ cells per well. Cells were washed with warm PBS and supplemented with 10% FBS media without antibiotics. Parallelly, bacterial cells were grown to late-log phase as described previously and were diluted in cell culture medium to represent multiplicity of infection (MOI) of 50:1 bacteria/cell. Media with bacteria was added to the cultured macrophages and schwannoma cells and placed in an incubator at 37° C. for 60 minutes. To determine invasiveness of the bacterial strains. cultured cells were washed in PBS and incubated in medium containing gentamicin sulfate (50 ug/mL) to kill any extracellular bacteria attached to cell surface, for 30 minutes. Cells were then rinsed 5× times with 1 to 2 mL PBS followed by addition of 0.2 mL of 0.1% Triton X-100 for 10 minutes to lyse the cells and detach adhered bacteria. LB broth (0.8 mL) was then added, and each sample was vigorously mixed to prepare homogenous suspensions for serial dilutions. 10-fold dilutions were prepared and plated on LB agar medium and incubated at 37° C. overnight to count colony-forming units (CFU).

Flow Cytometry

Tissues were harvested from the mice (n=3/group) and cells were dissociated using freshly prepared lysis buffer (125 U/mL collagenase type XI, 60 U/mL hyaluronidase type I-s, 60 U/mL, DNasel, and 450 U/L collagenase type I (Sigma-Aldrich) in PBS containing 20 mM Hepes) at 37° C. for 1 hr in a water bath with gently flicking every 10 minutes for proper homogenization and cell dissociation. Cell suspension was passed through a pre-wetted strainer 70 μm cells strainer (BD-Falcon). Cells were quantified by mixing 10 uL of the suspension with 10 uL trypan blue before loading on to hemocytometer. Cell suspensions were centrifuged at 2000 rpm for 10 minute at 4° C. to remove lysis buffer, washed and re-suspended in 1×PBS to maintain 10⁶ cell/100 uL. Cells were incubated in 2 uL of FC blocking agent (BD Biosciences) for 15 min at RT. Cells were washed with PBS and then incubated with fluorescence-labeled antibodies against cell surface markers or different immune markers for 1 hr in dark followed by wash steps with PBS and permeabilized with 2% paraformaldehyde (PFA solution) overnight. Antibodies to the following mouse immune markers were used for surface staining: CD45, F4/80, CD206, CD86, LY6G, NK1.1, NKp46, CD11b, CD11c, CD4, CD8, CD3, CD25. FACS and analysis were performed using FACSAria and LSRFortessa, using FACSDiva software (BD Bioscience) and FlowJo software. The utilized antibodies are supplied in supplementary Table 1.

Data Analysis

All data are presented as group mean±standard error of the mean (SEM). Data were analyzed with GraphPad Prism and Microsoft Excel. Repeated-measure analysis of variance (ANOVA) was utilized to compare tumor volumes and/or signals as described⁷⁴. One-way ANOVA was used to analyze cytokine expression and flowcytometric data. P<0.05 was accepted as significant.

### Example 1. Intratumoral Attenuated S. typhimurium Injection Suppresses Tumor Growth in Xenograft Human and Allograft Murine Schwannoma Models

We evaluated whether intratumoral (i.t.) injection of S. typhimurium can control growth of human (HEI-193 cell line) and murine (08031-9 cell line) schwannomas developing in the sciatic nerve of nu/nu immunocompromised and FVB/N immunocompetent mice, respectively. Two different strains of S. typhimurium were assessed: VNP20009 and ΔppGpp. Both strains are mutated attenuated forms of the wild type bacteria that have shown higher tumor tropism and an increased safety profile in preclinical studies^{19,32,33}, and for VNP20009, in clinical studies as well^{25,34}.

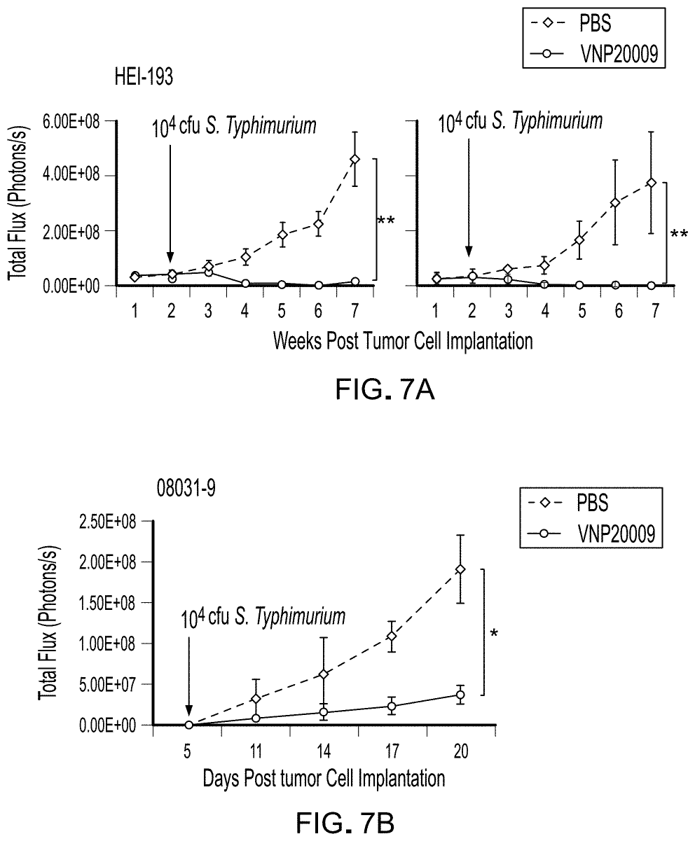

Tumor burden was assessed via in vivo bioluminescence imaging of firefly luciferase (Fluc) expressed by the HEI-193FC (human-NF2 schwannoma) and 08031-9FC (murine NF2-deficient schwannoma) cells. Once the tumor signals stabilized (about 2-weeks or 1-week following tumor implantation of HEI-193FC or 08031-9FC cells, respectively; FIG. 1A&B), tumor-bearing sciatic nerves were injected under direct visualization with attenuated S. typhimurium (VNP20009 or ΔppGpp) or PBS (control) (n=8 mice/group). Tumor growth was followed for additional 5-weeks in the human-NF2 schwannoma bearing nude mice, and for 2-weeks in the murine-NF2 schwannoma bearing immunocompetent FVB/N mice. In the xenograft schwannoma model studies were terminated 7-weeks following tumor cell implantation—a time at which most of the bacteria-injected tumors have no bioluminescent signal. Study termination in the intrasciatic allograft model was dictated by the development of motor dysfunction of the tumor-bearing hind limb of control mice (as assessed by both our group and an animal care technician, all of whom were blinded to the study groups). Two additional replications of this study were performed in the xenograft model and one replication in the allograft model; (n=8 mice/group for all studies; FIG. 7A&B).

Intratumoral injection of the VNP20009 strain of attenuated S. typhimurium led to a decrease in bioluminescent tumor signal compared to PBS control in all 3 replications in the xenograft human-NF2 schwannoma model (p<0.01, FIG. 1A; FIG. 7A) and both replications in the allograft mouse schwannoma model (p<0.05, FIG. 1B; FIG. 7B). The growth curves for bacterial and PBS injected mice begin to diverge in both tumor models as early as 1 week after VNP20009 injection. Bacterial treatment regressed HEI-193FC tumor signal to undetectable levels in 5 out of 8 mice by week-2 post bacterial injection (FIG. 1A). Across the 3 replicates of this experiment, 75% (18/24 mice) of the VNP20009-injected animals had no detectable tumor signal by the end of the experiment (FIG. 1A, FIG. 7A).

While we did not observe complete regression of tumor signal following bacteria injection in the allograft murine-schwannoma model, tumor growth control continued until the time of sacrifice when there was an approximate 8-fold lower bioluminescent signal in the VNP20009-injected mice compared to PBS controls (FIG. 1B, FIG. 7B).

We then tested whether the ΔppGpp strain of S. typhimurium would have similar therapeutic efficacy as VNP20009. While in the xenograft model the effect of VNP20009 and ΔppGpp on tumor signal were indistinguishable from one another, in the allograft mouse schwannoma model VNP20009, but not ΔppGpp strain of S. typhimurium controlled tumor growth (p<0.05 VNP20009 v. PBS, FIG. 1B), and in fact, there was a significant difference between the two strains (p<0.05 VNP20009 v. ΔppGpp, FIG. 1B).

Histological analysis of tumor-bearing nerves (n=3 mice/group) harvested at the end of the experiment (i.e., 5-weeks following bacterial injection in the xenograft human schwannoma model and 2-weeks following bacterial injection for the allograft mouse schwannoma model) showed abundant apoptotic bodies in both VNP20009 and ΔppGpp injected tumors, compared to the PBS-injected tumors (FIG. 1C&D). Quantification of tissues from the xenograft model revealed higher numbers of apoptotic bodies in both the VNP20009-injected (570±77; p<0.0005) and ΔppGpp-injected (230±68, p<0.005) schwannomas than the PBS injected tumors (4±1) (FIG. 1C). Comparison of apoptotic bodies induced by the 2 bacterial strains demonstrated greater numbers of apoptotic cells in the VNP20009-treated tumors compared to ΔppGpp-treated tumors in HEI-193 schwannomas. (p<0.01, FIG. 1C). There were approximately 3 times more apoptotic cells following VNP2009-injection than following ΔppGpp injection in the xenograft human NF-2 schwannomas. Concordant differences between groups were observed with the allograft mouse schwannoma model; there were more apoptotic cells in VNP20009-(340±63,) compared to ΔppGpp- (110±27, p<0.01) and PBS-injected tumors (6±0.3, p<0.001). ΔppGpp-injected tumors also significantly higher numbers of apoptotic cells than PBS controls (p<0.01, FIG. 1D). In this model i.t. VNP20009 injection led to approximately 3 times more apoptotic cells than ΔppGpp injection (p<0.01, FIG. 1D).

### Example 2. I.t. Attenuated S. typhimurium Injection Leads to Elevated Pro-Immunogenic Cytokines and Altered Immune Cell Infiltration in an Allograft Mouse-Schwannoma Model

One of the most exciting properties of BCT is its capacity to induce anti-tumor adaptive immunity³⁵⁻³⁷. Based on this finding we hypothesized that infection of schwannoma by attenuated S. typhimurium would have a vaccination effect inducing host anti-tumor adaptive immunity. Immunotherapy for schwannoma would be especially valuable given that affected individuals typically possess multiple tumors, develop new tumors throughout life, possess tumors in locations that cannot be surgically removed without substantial risk of major neuronal injury, and require multiple operations both because complete resection is often not feasible and, as mentioned, tumors arise throughout life.

While i.t. injection of bacteria into our xenograft and allograft schwannoma models leads to apoptotic cell death (FIG. 1), we wanted to investigate whether there is also evidence of pyroptotic and/or immunogenic cell death. Thus, we tested whether i.t. injection of VNP20009 and ΔppGpp into human HEI-193 and mouse 08031-9 schwannomas growing in the sciatic nerve of nude and immunocompetent mice, respectively, could induce broad indicators of host innate and adaptive immune responses. We evaluated intratumoral immune cell infiltration through analysis of the lymphocyte common antigen marker CD45³⁸ and the monocytes and tissue macrophages marker CD68³⁹. Immune cell infiltration was evaluated by immunocytochemical staining (n=3 tumors/treatment/model) in tumors collected 5-weeks post-bacteria injection in the xenograft human-NF2 model, and 2-weeks post-bacteria injection in the allograft mouse schwannoma model. Time of sacrifice was chosen for the reasons noted previously—i.e., resolution of tumor signal in most mice (xenograft model) or just prior to significant morbidity in control mice (allograft model).

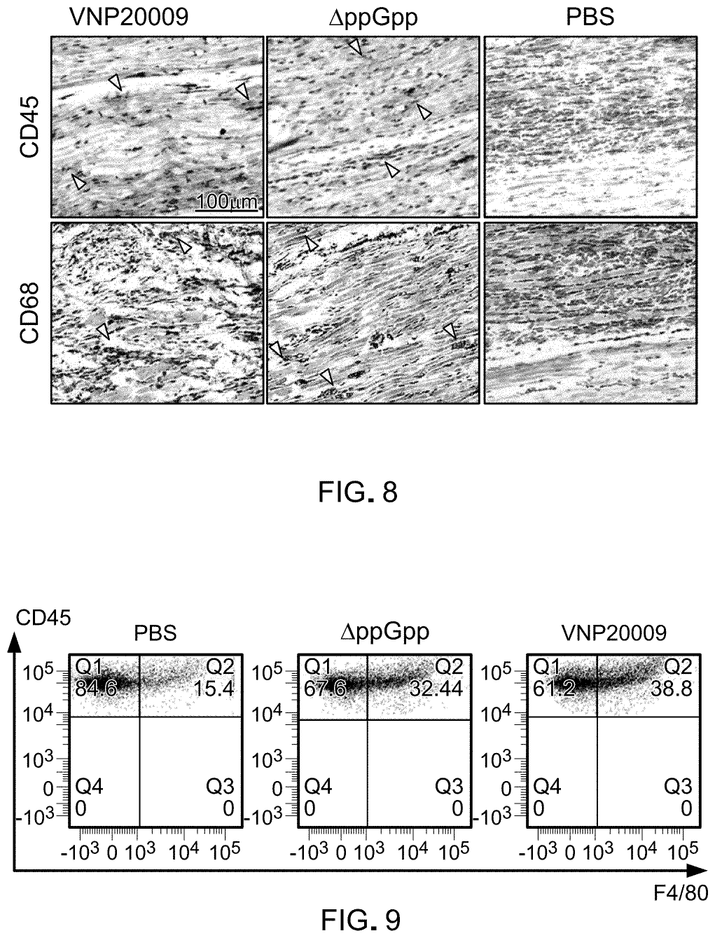

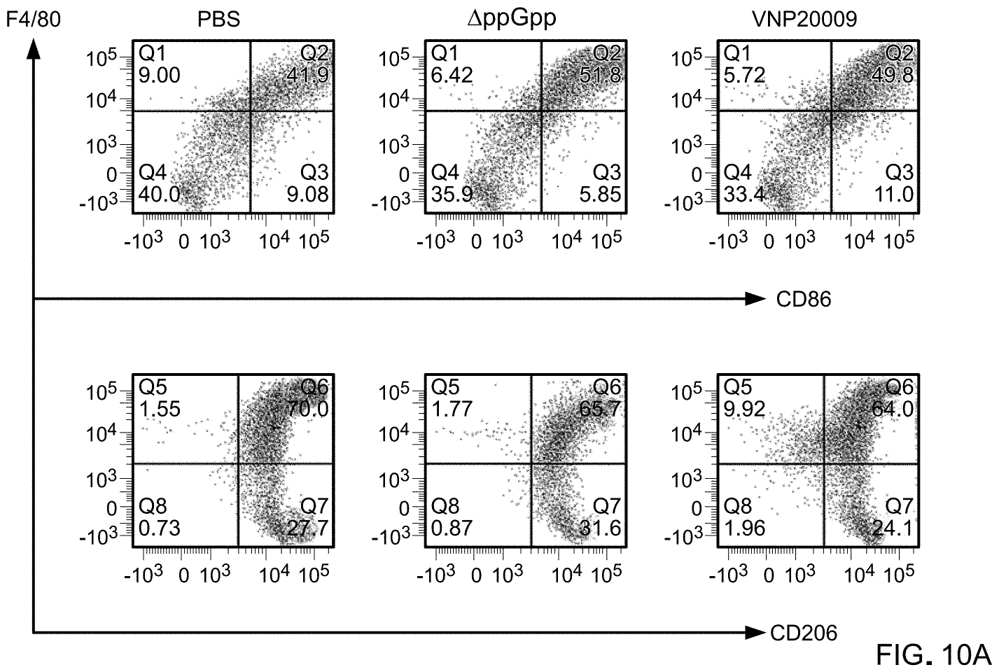

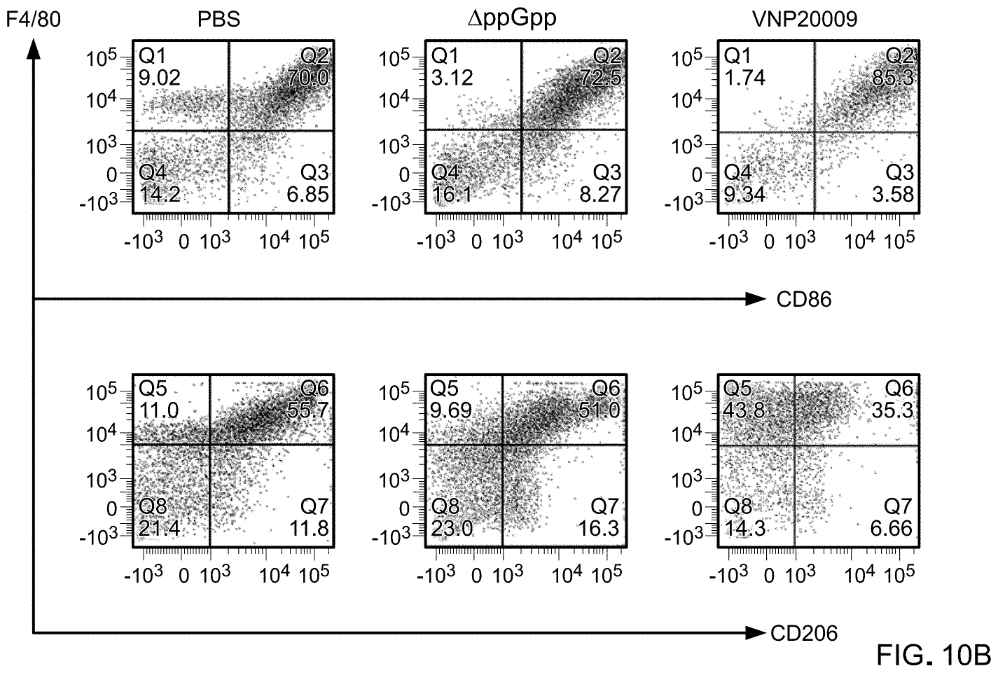

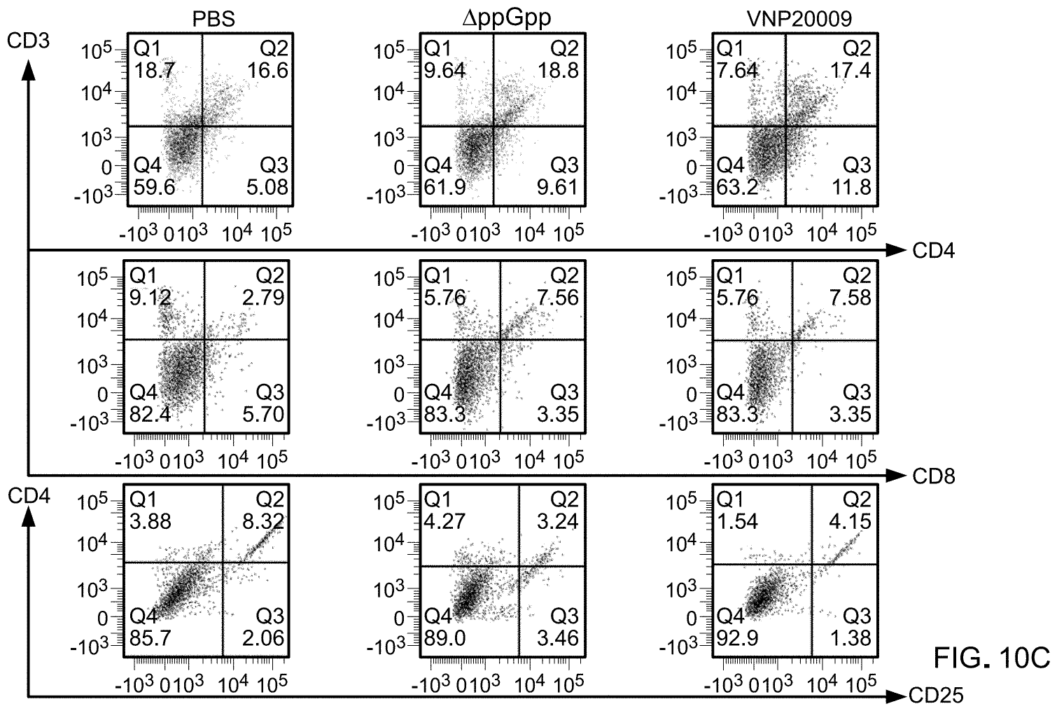

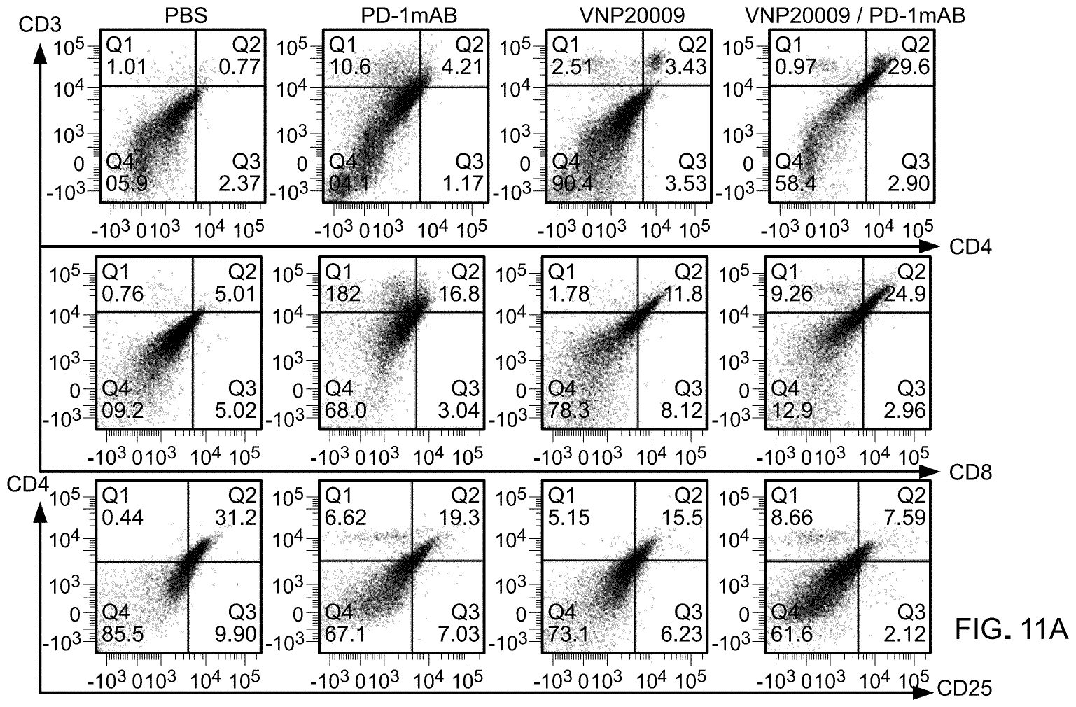

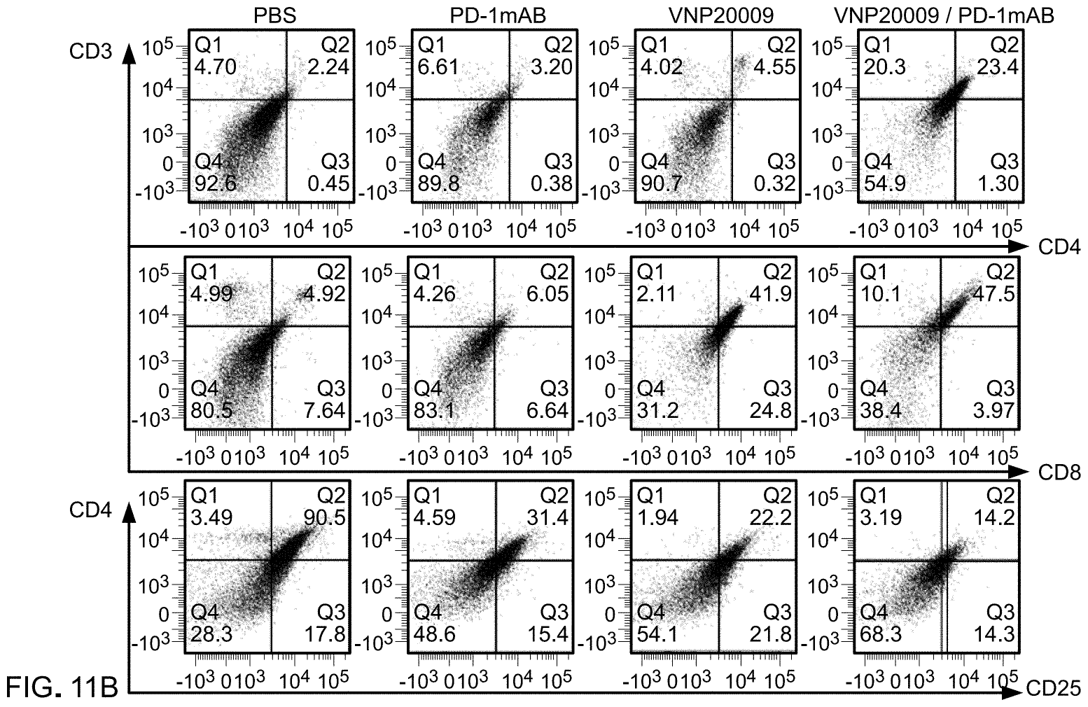

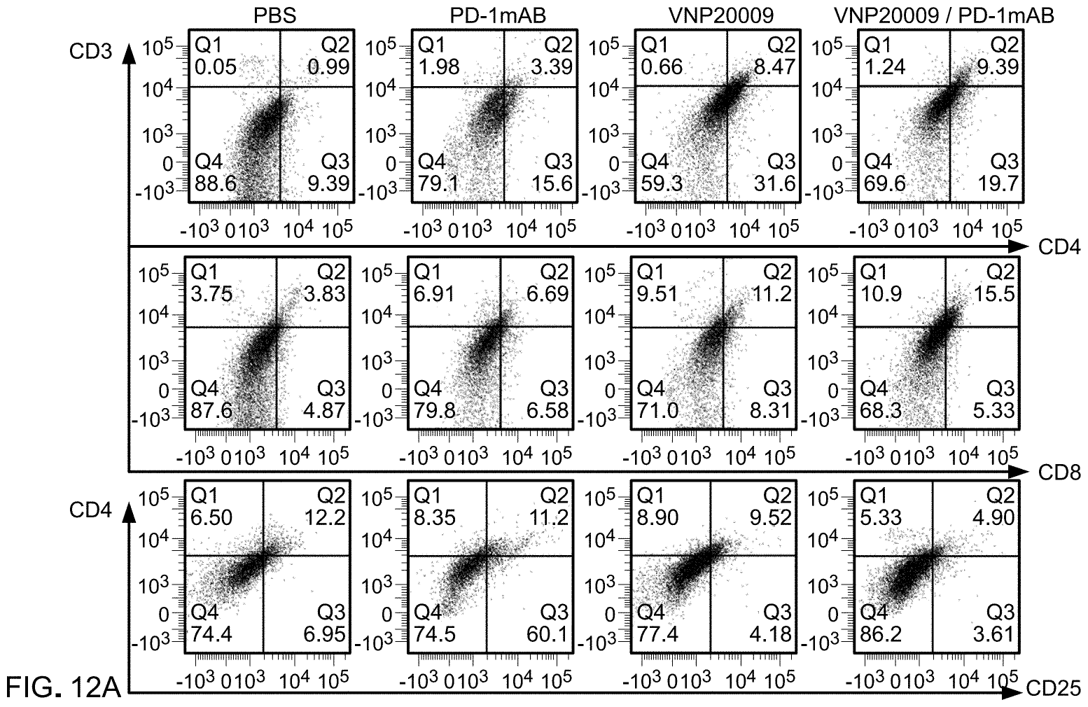

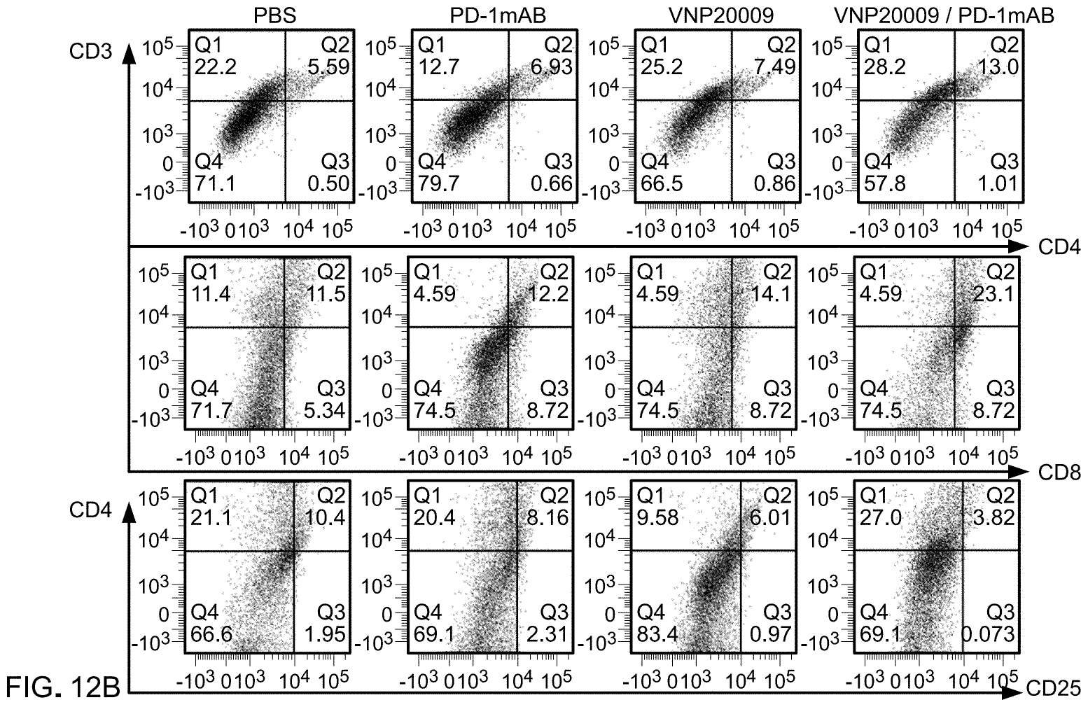

In the xenograft model, histological analysis of tumor-bearing nerves revealed abundant tumoral infiltration of CD45+ leukocytes and CD68+ macrophages compared to PBS injected tumors for which there was no indication of either class of cells (FIG. 8). The same analysis of intrasciatic allograft murine-schwannomas demonstrated that i.t. injection of either VNP20009 or ΔppGpp led to increased CD45+ leukocyte and CD68+ macrophages infiltration compared to PBS injected tumors (FIG. 2A). Quantification of the CD45+ and CD68+ cells in the tumor microenvironment of the allograft model showed that this bacterial injection resulted in increased tumor infiltrating leukocytes and macrophages compared to PBS injected controls (FIG. 2B).

Utilizing only the allograft model as these tumors develop in immunocompetent host mice, we then focused on characterizing S. typhimurium schwannoma killing to investigate whether there was indication of immunogenic cellular responses, as well as, immunogenic cell death. Macrophages can be categorized as Ml tumoricidal and M2 tumorigenic and appear to differentially be capable of promoting (M1)⁴⁰ or inhibiting (M2)⁴¹ host anti-tumor adaptive immunity, and a key determinant of host anti-tumor immunity is the balance of M1 and M2 macrophages. Human schwannomas are reported to be up composed of up to 50% macrophage by cell number⁴², and greater macrophage content has been associated with higher rates of tumor growth⁴³. We evaluated macrophage populations by flow cytometry of CD86+ (for M1-type, tumoricidal) and CD206+ (for M2-type, tumorigenic) expression in CD45+F4/80+ cells collected from intrasciatic allograft mouse schwannomas 3 and 7 days following i.t. injection of attenuated S. typhimurium. We found that i.t. injection of both VNP20009 and ΔppGpp shifted macrophage balance towards Ml type at 3-days post-bacteria injection increased the ratio of Ml to M2 macrophage (M1/M2) compared to PBS injection (p<0.05, FIG. 2C). At 7-days following i.t. bacterial injection the Ml/M2 ratio was further shifted towards Ml in VNP20009 injected tumors compared to PBS treated tumors (p<0.01, FIG. 2C), whereas in the ΔppGpp injected tumors the Ml/M2 ratio decreased compared to the 3-day timepoint and was no longer different from PBS (FIG. 2C). Interestingly, there were systemic effects of i.t. attenuated S. typhimurium injection on macrophage number. 3-days post i.t. bacterial injection splenic macrophages (CD45+F4/80+) were increased in VNP20009 (38.8%, p<0.01) and ΔppGpp (32.4%, p<0.01) groups compared to PBS (15.4%) (FIG. 9).

Given the observed increase in tumor infiltrating lymphocytes and the shift towards M1 type macrophages in bacterially injected allograft schwannomas, we investigated whether intratumoral T cell composition is altered by i.t. injection of attenuated S. typhimurium. Tumor infiltrating helper T cells (CD3/CD4), cytotoxic T cells (CD3/CD8) and regulatory T cells (Treg, CD4/CD25) was assessed using multi-colored flow cytometry 7-days following i.t. bacterial injection (n=3/group). While we did not observe an effect of i.t. bacterial injection on CD4+ helper T cells, i.t. injection of either VNP20009 or ΔppGpp increased the percentage of CD8+ cytotoxic T cells compared to PBS injection (7.56%, 7.56% and 2.79%, respectively, FIG. 2D). Further, i.t. VNP20009 or ΔppGpp injection reduced the number of tumor-infiltrating CD25+ Tregs, compared to PBS (4.15%, 3.24% and 8.32%, respectively, FIG. 2D). In all cases, these percentages represent proportion of CD45+ cells.

We then investigated the effects of i.t. VNP20009 and ΔppGpp injection on two key immunostimulatory cytokines, Tumor Necrosis Factor alpha (TNF-α and Interferon gamma (IFN-γ implicated in ICD and known to regulate the survival, proliferation and differentiation of both immune and tumors cells^{44,45}. We hypothesized that bacterial infection of schwannoma would induce production of these cytokines, in part because i.t. attenuated S. typhimurium leads to a shift towards M1 type macrophages (FIG. 2C). Moreover, S. typhimurium is a known inducer of inflammasomes (including NLRP3 and NLRC4)⁴⁶ that are involved in the processing and maturation of two pro-inflammatory cytokines, IL-1β and IL-18, known to possess anti-tumor activity^{19,47,48}. At day-3 post i.t. bacterial injection when VNP20009 and ΔppGpp mediated alteration in Ml/M2 ration was apparent (FIG. 2C), we observed elevation of multiple cytokines within intrasciatic allograft schwannomas. mRNA and proteins were extracted from tumors and cytokine profiles assessed using RT-PCR and ELISA (N=3/group). Transcript levels of the proinflammatory cytokines TNF-α, IFN-γ, IL-1β, and IL-18 were upregulated in VNP20009 and ΔppGpp S. typhimurium-injected tumors compared to controls (FIG. 2E). Of note, VNP20009-injected tumors showed higher TNF-α and IFN-γ mRNA expression levels compared to ΔppGpp (p<0.01, p<0.0001, respectively). As was observed at the transcript level; TNF-α, IFN-γ, IL-1β, and IL-18 protein levels were elevated in VNP20009 and ΔppGpp injected tumors compared to PBS controls (FIG. 2F). Further, i.t. VNP20009 injection led to greater IL-18, IFN-γ and TNF-α protein in the injected tumors compared to ΔppGpp (p<0.05, p<0.01, p<0.05, respectively; FIG. 2F). VNP20009 treatment also led to greater elevation of NLRC4 and NLRP3 mRNA compared to ΔppGpp or PBS (p<0.01, FIG. 2E).

### Example 3. I.t. S. typhimurium VNP20009 Injection Controls Growth of Bacterially-Injected and Contralateral Uninjected Allograft Mouse Schwannomas

A subset of schwannomas have been shown to contain PD-1 expressing CD4+ and CD8+ T cells indicating compromise of antitumor immunity in these cells⁴⁹. We found that i.t. VNP20009 injection of allograft schwannomas was associated with increased numbers of CD8+ cytotoxic T and decreased numbers of CD25+ Tregs suggesting activation of adaptive immune response. Thus, we evaluated combining systemic anti-PD-1 monoclonal antibody (mAb) with i.t. VNP20009 injection might enhance bacterially-induced host anti-tumor adaptive immune responses. FVB/N mice were subcutaneously implanted with 08031-9 mouse schwannoma cells into both flanks and divided into four groups (FIG. 3A shows experimental design): i) i.t. VNP20009 (left flank tumor), ii) i.p. anti-PD-1-mAb P, iii) i.t. VNP20009 and i.p. anti-PD-1-mAb, and iv) i.t. PBS (left tumor). Subcutaneous rather than intrasciatic implantation was utilized as the former allows longer survival, and thus, greater opportunity for adaptive immune responses to occur. Once mean tumor size reached approximately 150 mm^{3 50} (day-11 post implantation), VNP20009 (10⁴ CFU in 100 μl) or PBS was injected directly only in to the tumor implanted in the left flank. In parallel, anti-PD-1 mAb (250 μg/injection) was injected i.p. on days 10, 13, 16, and 19 following tumor-cell implantation⁵¹. We observed that compared to i.t. PBS injection, monotherapy with either VNP20009 or anti-PD-1 mAb suppressed growth of both tumors (FIG. 3B), and that i.t. VNP2009 led to greater growth control of the uninjected tumor than PD-1-mAb (p<0.05, FIG. 3B, D). Combination of i.t. VNP20009 with anti-PD-1 mAb led to 1) enhanced growth control of bacterially injected tumors compared to either VNP20009 or anti-PD-1 mAb alone (p<0.05, FIG. 3B), and 2) enhanced tumor growth control of uninjected contralateral tumors compared to anti-PD-1 mAb treatment (p<0.05) but no difference compared to VNP20009 treated mice (FIG. 3D).

These effects on schwannoma growth suggested that i.t. VNP20009 generated an adaptive immune response capable of controlling tumor growth and that this effect could be enhanced by immune checkpoint inhibition. Given this we analyzed T cell subsets in these tumors via flow cytometry—specifically, helper (CD3/CD4), cytotoxic (CD3/CD8) and regulatory T (CD4/CD25) T cells (FIG. 3C). In the left flank tumors (FIG. 3B,C) i.t. VNP20009 and systemic anti-PD-1 mAb led to 1) increased CD8+ cytotoxic T-cells (11.8% and 10.8%, respectively) compared to the PBS injection (1.01%), 2) increased CD4+ helper T cells (VNP20009 (3.43%); anti-PD-1 mAb (4.21%)) compared to PBS injection (0.77%), and 3) decreased in regulatory T cells (VNP20009 (15.5%), anti-PD-1 mAb (19.3%)), compared to PBS (31.2%). Combination of VNP20009 with anti-PD-1 mAb led to an additive increase of CD8+ (24.9%) and CD4+ (29.6%) T-cells compared to each monotherapy and PBS (FIG. 3C). There was also an additive effect of combination of bacterial and immune checkpoint inhibition on Treg depression (7.59%), compared to each monotherapy (VNP20009 (15.5%), anti-PD-1 mAb (19.3%), and PBS (31.2%).

To further evaluate whether these manipulations induced a systemic host anti-tumor immune response, we analyzed the same T cell populations in the right flank tumors which in no case underwent bacterial injection. I.t. VNP20009 (of the left flank tumor) combined with systemic anti-PD-1 mAb led to a synergistic effect in the uninjected tumor on infiltrating CD4+ helper T cells (23.4%), compared to VNP20009 (4.55%) or anti-PD-1 mAb (3.26%) alone; neither monotherapy was different from PBS (2.24%) (FIG. 3E). CD8+ cytotoxic T cell percentage in the right flank tumors was increased by either VNP20009/anti-PD-1 mAb combination (47.5%) or VNP20009 (41.9%) compared to both anti-PD-1 mAb (6.05%) and PBS treatment (4.92%), and VNP20009/anti-PD-1 mAb combination was greater than that of VNP20009 alone (FIG. 3E). Finally, as shown in FIG. 3E each treatment regimen reduced the percentage of tumor infiltrating CD25+ Tregs compared to i.t. PBS injection (of the contralateral left flank tumor) with the values as follows: VNP20009/anti-PD-1mAb (14.2%), VNP20009 (22.2%), anti-PD-1 mAb (31.4%) and PBS (50.5%). Of note, the effect on Treg reduction was greatest in the VNP20009/anti-PD-1mAb mice, and VNP20009 had a greater effect than anti-PD-1 mAb.

### Example 4. I.t. S. typhimurium (VNP20009) Injection of Primary Allograft Mouse Schwannomas Suppresses Growth of Bacterially-Uninjected Re-Challenge Schwannomas

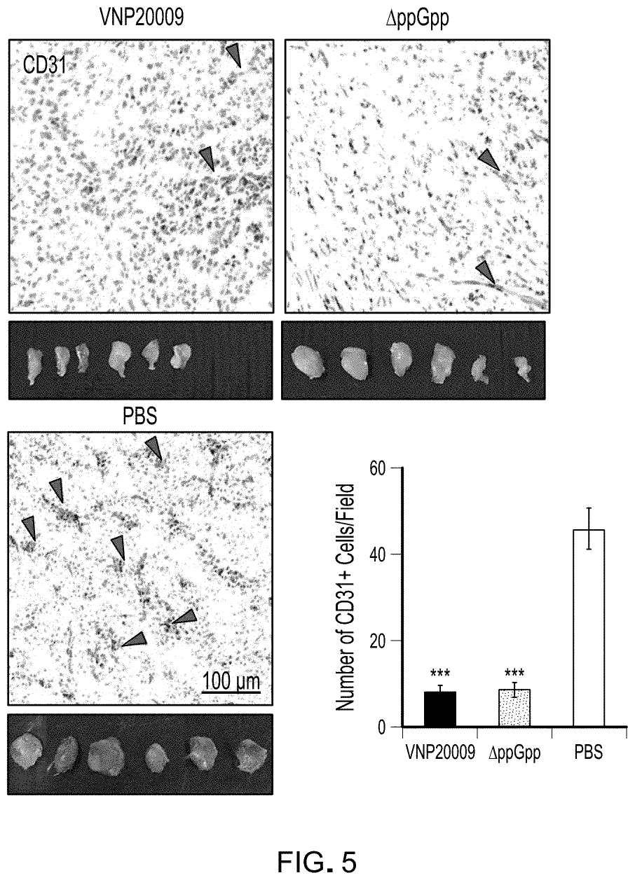

To investigate whether i.t. VNP20009 of schwannoma alone or in combination with anti-PD-1 mAb can generate a lasting anti-tumor adaptive immune response, we utilized a rechallenge model. 08031-9 mouse schwannoma cells were implanted in the left flank of FVB/N mice and as schematically shown in FIG. 5A divided into the following groups: i) i.t. VNP20009, ii) i.p. anti-PD-1 mAb P, iii) i.t. VNP20009 and i.p. anti-PD-1 mAb, and iv) i.t. PBS. I.t. VNP20009 (10⁴ CFU in 100 μl) was injected when average tumor size reached 150 mm^{3 50} (day-8 post implantation). Anti-PD-1 mAb (250 μg/injection)⁵¹ was administered i.p. on days 7, 10, 13, and 16 following tumor cell implantation. Replicating the results shown in FIG. 3, all treatment regimens, VNP20009/anti-PD-1 mAb, VNP20009, and anti-PD-1 mAb suppressed tumor growth compared to PBS, and there was an additive effect of combining VNP20009, with anti-PD-1 mAb (FIG. 4B). 12-days following bacterial injection of the s.c. tumor (and 13-days after the first application of immune checkpoint inhibitor) animals were re-challenged by implanting 08031-9FC schwannoma cells into the contralateral sciatic nerve. We chose intrasciatic location both because it is orthotopic and to bias against seeing an effect since these allograft schwannomas develop more rapidly within the nerve than subcutaneously. Intrasciatic tumor growth was monitored via bioluminescence imaging and revealed that compared to PBS controls tumor growth was inhibited in mice previously treated with VNP20009 or VNP20009/anti-PD-1 mAb with no difference between these two treatments (FIG. 4D). Previous treatment with anti-PD-1 mAb alone did not alter tumor growth compared to PBS (FIG. 4D). Notably, the magnitude of the suppressive effect of i.t. VNP20009 appears to be greater in the rechallenge tumors (FIG. 4D) than in the primary bacterially-injected schwannomas (FIG. 4B).

We again analyzed, by flowcytometry, T cell composition of injected and re-challenge tumors at the time of sacrifice. In the subcutaneous primary tumors, the percentage of infiltrating CD4+ helper T cell was increased by VNP20009 (8.47%), anti-PD-1 mAb (3.39%) and the combination of anti-PD-1 mAb with VNP20009 (9.39%) compared to the PBS injected controls (0.99%); VNP20009 had a greater effect than anti-PD1-mAb but there was no increased affect when checkpoint inhibition was added to bacteria compared with bacteria alone (FIG. 4C). CD8+ cytotoxic T cell percentage in the subcutaneous tumors was also increased by VNP20009 (11.2%), anti-PD-1 mAb (6.69%), as well as, combination of VNP20009 with anti-PD-1 mAb (15.5%) compared to the PBS injection (3.83%); in this case the increase was greater in bacterially-injected than immune checkpoint treated tumors, and combination therapy led to a greater induction of CD8+ T cells than bacteria alone (FIG. 4C). In contrast, compared to PBS controls (12.2%) CD25+ regulatory T cells proportion in the subcutaneous tumors were not altered by systemic anti-PD-1 mAb (11.2%), but were depressed by both i.t. VNP20009 injection (9.52%) and the combination of bacterial injection with checkpoint inhibition (4.90%; FIG. 4C). This suppressive effect of combination therapy on tumoral Tregs was greater than that of bacterial treatment alone (FIG. 4C).

Quantification of T-lymphocyte populations in the intrasciatic re-challenge tumors revealed a different pattern of treatment effects than that in the primary subcutaneous tumors. Compared to PBS control animals (5.59%), CD4+ helper T cell percentage was only increased in the tumors of mice previously exposed to the combination of VNP20009 and anti-PD-1 mAb (13%); there was no effect of prior VNP20009 (7.49%) or anti-PD-1 mAb monotherapy (6.93%) (FIG. 4E). In these rechallenge tumors, compared to PBS controls (11.5%) CD8+ cytotoxic T cell percentage was increased in mice previously treated with VNP20009 (14.1%) or VNP20009/anti-PD-1 mAb combination (23.1%); there was no effect of prior checkpoint inhibition alone (anti-PD-1 mAb, 12.2%; FIG. 4E). Similarly, CD25+ Treg percentage in re-challenge tumors was reduced by prior VNP20009 (6.01%) or VNP20009/anti-PD-1 mAb combination (3.82%) treatment, but not by anti-PD-1 mAb (8.16%) compared to PBS control mice (10.4%) (FIG. 4E). While there was no effect of prior immune checkpoint inhibition alone on Treg percentage in rechallenge schwannomas, the addition of anti-PD-1 mAb to VNP20009 enhanced the suppressive effect of prior bacterial treatment alone (FIG. 4E).

### Example 5. I.t. S. typhimurium Injection Suppresses Angiogenesis in Allograft Murine Schwannomas

S. typhimurium has been shown to reduce the expression of vascular endothelial growth factor (VEGF), which is an important pro-angiogenic factor⁵², and to have anti-angiogenic properties in preclinical cancer models^{24,53} Bevacizumab, an anti-angiogenic monoclonal antibody directed against VEGF-A, can control schwannoma growth in a subset of individuals with schwannoma⁵⁴. Given these observations, we investigated the effect of i.t. injection of attenuated S. typhimurium on tumor vascularity.

Intrasciatic mouse 08031-9 schwannomas evaluated 2-weeks following i.t. injection of attenuated S. typhimurium demonstrated inhibition of tumor angiogenesis compared to PBS-injected controls. Tumor angiogenesis was assessed by direct visualization and immunohistochemistry for the vascular endothelial marker CD31+⁵⁵ (FIG. 5). Macroscopic evaluation of tumors (N=6/mice group) showed an easily distinguishable difference between all the PBS-injected schwannomas which are bright red and have prominent external vascularity, compared to S. typhimurium-injected tumors which are pale in color with minimal or absent external vascularization (FIG. 5). Both VNP20009 and ΔppGpp injected tumors had diminished numbers of CD31+ cells (7.71±1.89, P<0.001 and 8.41±1.68, P<0.001, respectively) compared to PBS-injected tumors (45.71±4.75; FIG. 5, N=3/group).

### Example 6. I.t. S. typhimurium Injection Suppresses Growth of Subcutaneous Xenograft Human NF1, Human Sporadic MPNSTs, and Human Meningioma Tumors

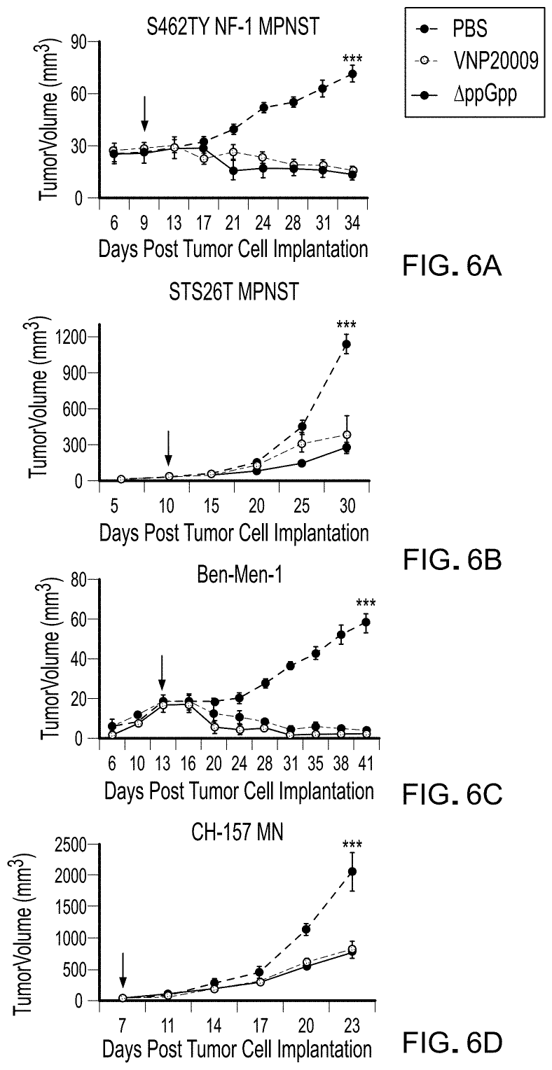

We also evaluated the effect of i.t. S. typhimurium (VNP20009 and ΔppGpp) injection on the growth of human NF-1 xenograft model in which immunocompromised nu/nu mice were subcutaneously implanted, in the left flank, with either human NF1-associated (S462TY, FIG. 6A) or sporadic (STS26, FIG. 6B) malignant peripheral nerve sheath tumor cells (MPNST)⁵⁶. Moreover, the efficacy of the i.t. S. typhimurium was attested in the subcutaneously implanted xenograft model in which nu/nu mice were implanted with benign meningioma (Ben-Men-1, FIG. 6C) or malignant meningioma (CH-157 MN, FIG. 6D) cell lines. In the tested model, mice were divided into 3 groups (n=5): VNP20009 or ΔppGpp (10⁴ CFU in 100 μl) vs. PBS injected control. Intra-tumoral injection was conducted once the tumor mass was macroscopically visible, and tumor growth was monitored by caliper measurement.

In the NF-1 xenograft model, our data showed that i.t. injection with either VNP20009 or ΔppGpp significantly suppressed tumor growth of the NF-1 associated S462TY MPNST cells (P<0.001, FIG. 6A) and controlled the tumor growth of the fast growing sporadic STS26T MPNST cells (P<0.001, FIG. 6B), compared to i.t. PBS injection. At the time of sacrifice (day 31 for S462TY and day 30 for STS26T), and compared to the PBS controls, the VNP20009 and ΔppGpp injected mice showed an approximate 7-fold and 4-fold lower tumor size in S462TY and STS26T models, respectively.

Similarly, in the meningioma xenograft model, i.t. injection of either VNP20009 or ΔppGpp significantly regressed tumor growth of the benign (Ben-Men-1, FIG. 6C) meningioma and controlled the tumor growth of the malignant (CH-157, FIG. 6D) meningioma, compared to the PBS control. In the benign model, 2 out of 5 mice showed complete regression at the day of sacrifice (day 38) in the mice which were injected with either VNP20009 or ΔppGpp S. typhimurium. At the day of sacrifice of the malignant meningioma, there was an approximate 3-fold lower tumor size in the VNP20009 and ΔppGpp injected mice, compared to the PBS controls. The experiment was stopped at day 23 when tumors in the control group became ulcerated/necrotic.

### REFERENCES

1. Lu-Emerson C, Plotkin S R. The neurofibromatoses. Part 2: NF2 and schwannomatosis. Rev Neurol Dis. 2009; 6(3):E81-86.
2. Antinheimo J, Sankila R, Carpen O, Pukkala E, Sainio M, Jaaskelainen J. Population-based analysis of sporadic and type 2 neurofibromatosis-associated meningiomas and schwannomas. Neurology. 2000; 54(1):71-76.
3. Evans D G. Neurofibromatosis type 2 (NF2): a clinical and molecular review. Orphanet J Rare Dis. 2009; 4:16.
4. Merker V L, Esparza S, Smith M J, Stemmer-Rachamimov A, Plotkin S R. Clinical features of schwannomatosis: a retrospective analysis of 87 patients. Oncologist. 2012; 17(10):1317-1322.
5. Inoue H, Nakagawa Y, Ikemura M, Usugi E, Kiyofuji Y, Nata M. Acute brainstem compression by intratumoral hemorrhages in an intracranial hypoglossal schwannoma. Leg Med (Tokyo). 2013; 15(5):249-252.
6. Korf B R. Neurofibromatosis. Handb Clin Neurol. 2013; 111:333-340.
7. Fehlings M G, Nater A, Zamorano J J, et al. Risk Factors for Recurrence of Surgically Treated Conventional Spinal Schwannomas: Analysis of 169 Patients From a Multicenter International Database. Spine (Phila Pa. 1976). 2016; 41(5):390-398.
8. Wong H K, Landenranta J, Kamoun W S, et al. Anti-vascular endothelial growth factor therapies as a novel therapeutic approach to treating neurofibromatosis-related tumors. Cancer Res. 2010; 70(9):3483-3493.
9. Lafayette R A, McCall B, Li N, et al. Incidence and relevance of proteinuria in bevacizumab-treated patients: pooled analysis from randomized controlled trials. Am J Nephrol. 2014; 40(1):75-83.
10. Bakker A C, La Rosa S, Sherman L S, et al. Neurofibromatosis as a gateway to better treatment for a variety of malignancies. Prog Neurobiol. 2017; 152:149-165.
11. Taugourdeau-Raymond S, Rouby F, Default A, Jean-Pastor M J, French Network of Pharmacovigilance C. Bevacizumab-induced serious side-effects: a review of the French pharmacovigilance database. Eur J Clin Pharmacol. 2012; 68(7):1103-1107.
12. Coley W B. The treatment of malignant tumors by repeated inoculations of erysipelas. With a report of ten original cases. 1893. Clin Orthop Relat Res. 1991(262):3-11.
13. Min J J, Kim H J, Park J H, et al. Noninvasive real-time imaging of tumors and metastases using tumor-targeting light-emitting Escherichia coli. Mol Imaging Biol. 2008; 10(1):54-61.
14. Min J J, Nguyen V H, Kim H J, Hong Y, Choy H E. Quantitative bioluminescence imaging of tumor-targeting bacteria in living animals. Nat Protoc. 2008; 3(4):629-636.
15. Yu Y A, Shabahang S, Timiryasova T M, et al. Visualization of tumors and metastases in live animals with bacteria and vaccinia virus encoding light-emitting proteins. Nat Biotechnol. 2004; 22(3):313-320.
16. Zhao M, Geller J, Ma H, Yang M, Penman S, Hoffman R M. Monotherapy with a tumor-targeting mutant of Salmonella typhimurium cures orthotopic metastatic mouse models of human prostate cancer. Proc Natl Acad Sci USA. 2007; 104(24): 10170-10174.
17. Zhao M, Yang M, Li X M, et al. Tumor-targeting bacterial therapy with amino acid auxotrophs of GFP-expressing Salmonella typhimurium. Proc Natl Acad Sci USA. 2005; 102(3):755-760.
18. Wen M, Jung S, Moon K S, Jiang S N, Li S Y, Min J J. Targeting Orthotopic Glioma in Mice with Genetically Engineered Salmonella typhimurium. J Korean Neurosurg Soc. 2014; 55(3):131-135.
19. Kim J E, Phan T X, Nguyen V H, et al. Salmonella typhimurium Suppresses Tumor Growth via the Pro-Inflammatory Cytokine Interleukin-1beta. Theranostics. 2015; 5(12):1328-1342.
20. Zheng J H, Nguyen V H, Jiang S N, et al. Two-step enhanced cancer immunotherapy with engineered Salmonella typhimurium secreting heterologous flagellin. Sci Transl Med. 2017; 9(376).
21. Miyake K, Kawaguchi K, Miyake M, et al. Tumor-targeting Salmonella typhimurium A1-R suppressed an imatinib-resistant gastrointestinal stromal tumor with c-kit exon 11 and 17 mutations. Heliyon. 2018; 4(6): e00643.
22. Zheng J H, Min J J. Targeted Cancer Therapy Using Engineered Salmonella typhimurium. Chonnam Med J. 2016; 52(3):173-184.
23. Tu D G, Chang W W, Lin S T, Kuo C Y, Tsao Y T, Lee C H. Salmonella inhibits tumor angiogenesis by downregulation of vascular endothelial growth factor. Oncotarget. 2016; 7(25):37513-37523.
24. Kiyuna T, Tome Y, Uehara F, et al. Tumor-targeting Salmonella typhimurium A1-R Inhibits Osteosarcoma Angiogenesis in the In Vivo Gelfoam(R) Assay Visualized by Color-coded Imaging. Anticancer Res. 2018; 38(1):159-164.
25. Toso J F, Gill V J, Hwu P, et al. Phase I study of the intravenous administration of attenuated Salmonella typhimurium to patients with metastatic melanoma. J Clin Oncol. 2002; 20(1):142-152.
26. Nemunaitis J, Cunningham C, Senzer N, et al. Pilot trial of genetically modified, attenuated Salmonella expressing the E. coli cytosine deaminase gene in refractory cancer patients. Cancer Gene Ther. 2003; 10(10):737-744.
27. Kaimala S, Al-Sbiei A, Cabral-Marques O, Fernandez-Cabezudo M J, Al-Ramadi B K. Attenuated Bacteria as Immunotherapeutic Tools for Cancer Treatment. Front Oncol. 2018; 8:136.
28. Singh R, Wallecha A. Cancer immunotherapy using recombinant Listeria monocytogenes: transition from bench to clinic. Hum Vaccin. 2011; 7(5):497-505.
29. Swietek N, Waldert M, Susani M, Schatzl G, Klatte T. Intravesical bacillus Calmette-Guerin instillation therapy for non-muscle-invasive bladder cancer following solid organ transplantation. Wien Klin Wochenschr. 2013; 125(7-8):189-195.
30. Chorobik P, Czaplicki D, Ossysek K, Bereta J. Salmonella and cancer: from pathogens to therapeutics. Acta Biochim Pol. 2013; 60(3):285-297.
31. Low K B, Ittensohn M, Le T, et al. Lipid A mutant Salmonella with suppressed virulence and TNFalpha induction retain tumor-targeting in vivo. Nat Biotechnol. 1999; 17(1):37-41.
32. Han Y H, Lai X H, Le Z W, Hua Z C. [Anti-tumor effect and impact on tumor immune microenvironment of tumor-targeted Salmonella VNP20009]. Yao Xue Xue Bao. 2016; 51(9):1417-1422.
33. Na H S, Kim H J, Lee H C, Hong Y, Rhee J H, Choy H E. Immune response induced by Salmonella typhimurium defective in ppGpp synthesis. Vaccine. 2006; 24(12):2027-2034.
34. Cunningham C, Nemunaitis J. A phase I trial of genetically modified Salmonella typhimurium expressing cytosine deaminase (TAPET-CD, VNP20029) administered by intratumoral injection in combination with 5-fluorocytosine for patients with advanced or metastatic cancer. Protocol no: CL-017. Version: Apr. 9, 2001. Hum Gene Ther. 2001; 12(12):1594-1596.
35. Patyar S, Joshi R, Byrav D S, Prakash A, Medhi B, Das B K. Bacteria in cancer therapy: a novel experimental strategy. J Biomed Sci. 2010; 17(1):21.
36. Dougan M, Dougan S K. Programmable bacteria as cancer therapy. Nat Med. 2019; 25(7):1030-1031.
37. Sarotra P, Medhi B. Use of Bacteria in Cancer Therapy. Recent Results Cancer Res. 2016; 209:111-121.
38. Woodford-Thomas T, Thomas M L. The leukocyte common antigen, CD45 and other protein tyrosine phosphatases in hematopoietic cells. Semin Cell Biol. 1993; 4(6):409-418.
39. Barros M H, Hauck F, Dreyer J H, Kempkes B, Niedobitek G. Macrophage polarisation: an immunohistochemical approach for identifying M1 and M2 macrophages. PLoS One. 2013; 8(11):e80908.
40. Jarosz-Biej M, Kaminska N, Matuszczak S, et al. M I-like macrophages change tumor blood vessels and microenvironment in murine melanoma. PLoS One. 2018; 13(1):e0191012.
41. Suarez-Lopez L, Sriram G, Kong Y W, et al. MK2 contributes to tumor progression by promoting M2 macrophage polarization and tumor angiogenesis. Proc Natl Acad Sci USA. 2018; 115(18):E4236-E4244.
42. de Vries M, Briaire-de Bruijn I, Malessy M J, de Bruine S F, van der Mey A G, Hogendoorn P C. Tumor-associated macrophages are related to volumetric growth of vestibular schwannomas. Otol Neurotol. 2013; 34(2):347-352.
43. Lewis D, Roncaroli F, Agushi E, et al. Inflammation and vascular permeability correlate with growth in sporadic vestibular schwannoma. Neuro Oncol. 2019; 21(3):314-325.
44. Wajant H. The role of TNF in cancer. Results Probl Cell Differ. 2009; 49:1-15.
45. Ni L, Lu J. Interferon gamma in cancer immunotherapy. Cancer Med. 2018; 7(9):4509-4516.
46. Crowley S M, Knodler L A, Vallance B A. Salmonella and the Inflammasome: Battle for Intracellular Dominance. Curr Top Microbiol Immunol. 2016; 397:43-67.
47. Phan T X, Nguyen V H, Duong M T, Hong Y, Choy H E, Min J J. Activation of inflammasome by attenuated Salmonella typhimurium in bacteria-mediated cancer therapy. Microbiol Immunol. 2015; 59(11):664-675.
48. Sollberger G, Strittmatter G E, Garstkiewicz M, Sand J, Beer H D. Caspase-1: the inflammasome and beyond. Innate Immun. 2014; 20(2):115-125.
49. Wang S, Liechty B, Patel S, et al. Programmed death ligand 1 expression and tumor infiltrating lymphocytes in neurofibromatosis type 1 and 2 associated tumors. J Neurooncol. 2018; 138(1):183-190.
50. Tanaka K, Eskin A, Chareyre F, et al. Therapeutic potential of HSP90 inhibition for neurofibromatosis type 2. Clin Cancer Res. 2013; 19(14):3856-3870.
51. Wang S, Campos J, Gallotta M, et al. Intratumoral injection of a CpG oligonucleotide reverts resistance to PD-1 blockade by expanding multifunctional CD8+ T cells. Proc Natl Acad Sci USA. 2016; 113(46):E7240-E7249.
52. Carmeliet P. VEGF as a key mediator of angiogenesis in cancer. Oncology. 2005; 69 Suppl 3:4-10.
53. Liu F, Zhang L, Hoffman R M, Zhao M. Vessel destruction by tumor-targeting Salmonella typhimurium A1-R is enhanced by high tumor vascularity. Cell Cycle. 2010; 9(22):4518-4524.
54. Lu V M, Ravindran K, Graffeo C S, et al. Efficacy and safety of bevacizumab for vestibular schwannoma in neurofibromatosis type 2: a systematic review and meta-analysis of treatment outcomes. J Neurooncol. 2019; 144(2):239-248.
55. Majchrzak K, Kaspera W, Szymas J, Bobek-Billewicz B, Hebda A, Majchrzak H. Markers of angiogenesis (CD31, CD34, rCBV) and their prognostic value in low-grade gliomas. Neurol Neurochir Pol. 2013; 47(4):325-331.
56. Sane K M, Mynderse M, Lalonde D T, et al. A novel geranylgeranyl transferase inhibitor in combination with lovastatin inhibits proliferation and induces autophagy in STS-26T MPNST cells. J Pharmacol Exp Ther. 2010; 333(1):23-33.
57. Kaimala S, Mohamed Y A, Nader N, et al. Salmonella-mediated tumor regression involves targeting of tumor myeloid suppressor cells causing a shift to M1-like phenotype and reduction in suppressive capacity. Cancer Immunol Immunother. 2014; 63(6):587-599.
58. Thamm D H, Kurzman I D, King I, et al. Systemic administration of an attenuated, tumor-targeting Salmonella typhimurium to dogs with spontaneous neoplasia: phase I evaluation. Clin Cancer Res. 2005; 11(13):4827-4834.
59. Wang Y, Li P, Wang B, Wang S, Liu P. Identification of myeloid-derived suppressor cells that have an immunosuppressive function in NF2 patients. J Cancer Res Clin Oncol. 2019; 145(2):523-533.
60. Zhang M, He Y, Sun X, et al. A high M1/M2 ratio of tumor-associated macrophages is associated with extended survival in ovarian cancer patients. J Ovarian Res. 2014; 7:19.
61. He Y F, Zhang M Y, Wu X, et al. High MUC2 expression in ovarian cancer is inversely associated with the M1/M2 ratio of tumor-associated macrophages and patient survival time. PLoS One. 2013; 8(12):e79769.
62. Italiani P, Boraschi D. From Monocytes to M1/M2 Macrophages: Phenotypical vs. Functional Differentiation. Front Immunol. 2014; 5:514.
63. Fairweather D, Cihakova D. Alternatively activated macrophages in infection and autoimmunity. J Autoimmun. 2009; 33(3-4):222-230.
64. Mantovani A, Sica A, Sozzani S, Allavena P, Vecchi A, Locati M. The chemokine system in diverse forms of macrophage activation and polarization. Trends Immunol. 2004; 25(12):677-686.
65. Sindrilaru A, Peters T, Wieschalka S, et al. An unrestrained proinflammatory M1 macrophage population induced by iron impairs wound healing in humans and mice. J Clin Invest. 2011; 121(3):985-997.
66. Anker J F, Naseem A F, Mok H, Schaeffer A J, Abdulkadir S A, Thumbikat P. Multi-faceted immunomodulatory and tissue-tropic clinical bacterial isolate potentiates prostate cancer immunotherapy. Nat Commun. 2018; 9(1):1591.
67. Bonaventura P, Shekarian T, Alcazer V, et al. Cold Tumors: A Therapeutic Challenge for Immunotherapy. Front Immunol. 2019; 10:168.
68. Hung G, Li X, Faudoa R, et al. Establishment and characterization of a schwannoma cell line from a patient with neurofibromatosis 2. Int J Oncol. 2002; 20(3):475-482.
69. Lepont P, Stickney J T, Foster L A, Meng J J, Hennigan R F, Ip W. Point mutation in the NF2 gene of HEI-193 human schwannoma cells results in the expression of a merlin isoform with attenuated growth suppressive activity. Mutat Res. 2008; 637(1-2):142-151.
70. Prabhakar S, Brenner G J, Sung B, et al. Imaging and therapy of experimental schwannomas using HSV amplicon vector-encoding apoptotic protein under Schwann cell promoter. Cancer Gene Ther. 2010; 17(4):266-274.
71. Daigneault M, Preston J A, Marriott H M, Whyte M K, Dockrell D H. The identification of markers of macrophage differentiation in PMA-stimulated THP-1 cells and monocyte-derived macrophages. PLoS One. 2010; 5(1):e8668.
72. Saydam O, Ozdener G B, Senol 0, et al. A novel imaging-compatible sciatic nerve schwannoma model. J Neurosci Methods. 2011; 195(1):75-77.
73. Messerli S M, Tang Y, Giovannini M, Bronson R, Weissleder R, Breakefield X O. Detection of spontaneous schwannomas by MRI in a transgenic murine model of neurofibromatosis type 2. Neoplasia. 2002; 4(6):501-509.
74. Harris J E, Sheean P M, Gleason P M, Bruemmer B, Boushey C. Publishing nutrition research: a review of multivariate techniques—part 2: analysis of variance. J Acad Nutr Diet. 2012; 112(1):90-98.
75. E. R. Stanley, V. Chitu, CSF-1 receptor signaling in myeloid cells. Cold Spring Harbor perspectives in biology 6, (2014); published online EpubJun 2 (10.1101/cshperspect.a021857).
76. M. B. Pedersen, A. V. Danielsen, S. J. Hamilton-Dutoit, K. Bendix, P. Norgaard, M. B. Moller, T. Steiniche, F. d'Amore, High intratumoral macrophage content is an adverse prognostic feature in anaplastic large cell lymphoma. Histopathology 65, 490-500 (2014); published online EpubOct (10.1111/his.12407).
77. J. C. Lenzo, A. L. Turner, A. D. Cook, R. Vlahos, G. P. Anderson, E. C. Reynolds, J. A. Hamilton, Control of macrophage lineage populations by CSF-1 receptor and GM-CSF in homeostasis and inflammation. Immunology and cell biology 90, 429-440 (2012); published online EpubApr (10.1038/icb.2011.58).
78. D. Achkova, J. Maher, Role of the colony-stimulating factor (CSF)/CSF-1 receptor axis in cancer. Biochemical Society transactions 44, 333-341 (2016); published online EpubApr 15 (10.1042/BST20150245).
79. Q. W. Zhang, L. Liu, C. Y. Gong, H. S. Shi, Y. H. Zeng, X. Z. Wang, Y. W. Zhao, Y. Q. Wei, Prognostic significance of tumor-associated macrophages in solid tumor: a meta-analysis of the literature. PloS one 7, e50946 (2012)10.1371/journal.pone.0050946).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of a treating a subject having or at risk of having a benign nervous system tumor, the method comprising administering to the subject a therapeutically effective amount of a composition comprising live attenuated Salmonella bacteria, optionally in combination with an immune checkpoint inhibitor and/or angiogenesis inhibitor.

2. The method of claim 1, wherein the subject is a subject having or diagnosed as having a benign tumor or tumor-associated condition selected from the group consisting of: neurofibromatosis 1 (NF1); neurofibromatosis 2 (NF2); schwannomatosis; meningioma; schwannoma; vestibular schwannoma; sporadic schwannoma; neurofibroma; neurofibromatosis (NF); or any combination thereof.

3. The method of claim 1, wherein the subject does not have a malignant solid tumor.

4. The method of claim 1, wherein the subject has a condition associated with an increased risk of a benign nervous system tumor.

5. The method of claim 4, wherein the condition associated with an increased risk of a benign nervous system tumor is neurofibromatosis 1 (NF1); neurofibromatosis 2 (NF2); or schwannomatosis.

6. The method of claim 1, wherein the attenuated Salmonella is administered intratumorally or intravenously.

7. The method of claim 1, wherein the attenuated Salmonella is an attenuated strain of S. typhimurium.

8. The method of claim 7, wherein the attenuated strain of S. typhimurium is Salmonella enterica serovar typhimurium strain VNP20009 with modified lipid A (msbB−) and purine auxotrophic mutation (purI−).

9. The method of claim 1, wherein the composition does not comprise Clostridium novyi.

10. The method of claim 1, wherein the attenuated Salmonella do not comprise a lysis gene or cassette operably linked to an intracellularly induced Salmonella promoter.

11. The method of claim 1, wherein the checkpoint inhibitor is an inhibitor of PD-1 or CTLA-4 signaling.

12. The method of claim 11, wherein the inhibitor of PD-1 signaling is an antibody that binds to PD-1, CD40, PD-L1, or CTLA-4.

13. The method of claim 1, wherein the angiogenesis inhibitor is an inhibitor of vascular endothelial growth factor (VEGF) or its receptor (VEGFR).

14. The method of claim 11, wherein the inhibitor of VEGF is Bevacizumab.

15. A composition comprising live attenuated Salmonella bacteria in combination with a checkpoint inhibitor and/or angiogenesis inhibitor.

16. (canceled)

17. (canceled)

18. (canceled)

19. (canceled)

20. The composition of claim 15, wherein the attenuated Salmonella is formulated to be administered intratumorally or intravenously.

21. The composition of claim 15, wherein the attenuated Salmonella is an attenuated strain of S. typhimurium.

22. The composition of claim 21, wherein the attenuated strain of S. typhimurium is Salmonella enterica serovar typhimurium strain VNP20009 with modified lipid A (msbB−) and purine auxotrophic mutation (purI−).

23. The composition of claim 15, wherein the composition does not comprise Clostridium novyi.

24. The composition of claim 15, wherein the attenuated Salmonella do not comprise a lysis gene or cassette operably linked to an intracellularly induced Salmonella promoter.

25. The composition of claim 15, wherein the checkpoint inhibitor is an inhibitor of PD-1 or CTLA-4 signaling.

26. The composition of claim 25, wherein the inhibitor of PD-1 or CTLA-4 signaling is an antibody that binds to PD-1, CD40, PD-L1, or CTLA-4.

27. The composition of claim 15, wherein the angiogenesis inhibitor is an inhibitor of vascular endothelial growth factor (VEGF) or its receptor (VEGFR).

28. The composition of claim 15, wherein the inhibitor of VEGF is Bevacizumab.
